SEQUENCE LISTING (11 sequences)

SEQ ID NO: 1 - protein, 173 aa, synthetic construct
AEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSR

SEQ ID NO: 2 - protein, 154 aa, synthetic construct
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 182 aa, synthetic construct
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 4 - DNA, 20 bp, synthetic construct
  source 1..20: mol_type = other DNA
gcgagaaccagcaagatgta

SEQ ID NO: 5 - DNA, 20 bp, synthetic construct
  source 1..20: mol_type = other DNA
gcttggtgactgtggtgtat

SEQ ID NO: 6 - DNA, 20 bp, synthetic construct
  source 1..20: mol_type = other DNA
gtaacccgttgaaccccatt

SEQ ID NO: 7 - DNA, 20 bp, synthetic construct
  source 1..20: mol_type = other DNA
ccatccaatcggtagtagcg

SEQ ID NO: 8 - DNA, 22 bp, synthetic construct
  source 1..22: mol_type = other DNA
ccctaactttcccaatcctctg

SEQ ID NO: 9 - DNA, 22 bp, synthetic construct
  source 1..22: mol_type = other DNA
gcttgcagcggacaataaataa

SEQ ID NO: 10 - DNA, 20 bp, synthetic construct
  source 1..20: mol_type = other DNA
tgcatggccgttcttagttg

SEQ ID NO: 11 - DNA, 23 bp, synthetic construct
  source 1..23: mol_type = other DNA
agttagcatgccagagtctcgtt

(19) United States
(12) Patent Application Publication
(10) Pub. No.: US 2026/0199345 A1
(43) Pub. Date: Jul. 16, 2026

(54) SARCOPENIA TREATMENT EFFECT THROUGH INHIBITION OF FGF18 SIGNALING PATHWAY

(71) Applicant: KNU-INDUSTRY COOPERATION FOUNDATION, Chuncheon-si (KR)

(72) Inventors: Yong-Hyun HAN, Seoul (KR); Dong-Hyun KIM, Seoul (KR)

(73) Assignee: KNU-INDUSTRY COOPERATION FOUNDATION, Chuncheon-si (KR)

(21) Appl. No.: 19/437,977

(22) Filed: Dec. 31, 2025

(30) Foreign Application Priority Data
Jan. 2, 2025 (KR) 10-2025-0000532

(51) Int. Cl. A61K 31/517; A61P 21/00; C12Q 1/6883; G01N 33/68

(52) U.S. Cl. CPC A61K 31/517; A61P 21/00; C12Q 1/6883; G01N 33/6872; C12Q 2600/106; C12Q 2600/158; G01N 2333/50; G01N 2800/10; G01N 2800/52

(57) ABSTRACT

Provided are a method for treating a muscle weakness-related disease by using an agent that inhibits the expression of a fibroblast growth factor receptor 4 (FGFR4) gene or the activity of FGFR4 protein, and a method of characterizing a responder to a therapeutic agent for a muscle weakness-related disease and a method of treating the responder by measuring an expression level of a FGF18 protein or an mRNA thereof in a biological sample, determining a responder to the therapeutic agent when the expression level of the FGF18 protein or mRNA increases, compared with a control, and administering to the responder a pharmaceutically effective amount of the agent that treats a muscle weakness-related disease.

### REFERENCE TO AN ELECTRONIC SEQUENCE LISTING

The instant application contains a Sequence Listing which has been submitted electronically in XML format and is hereby incorporated by reference in its entirety. Said XML copy, created on Dec. 31, 2025, is named SequenceListing.xml and is 11,237 bytes in size.

### TECHNICAL FIELD

The present invention relates to a sarcopenia treatment effect through inhibition of the FGF18 signaling pathway.

### BACKGROUND ART

Skeletal muscle is the largest organ in the human body, accounting for 40 to 50% of the total body weight, and plays a crucial role in various metabolic functions in the body, including energy homeostasis and thermogenesis. As the human body ages, changes in composition lead to a redistribution of body fat and protein. Around age 50, the rate of protein synthesis within muscle cells slows down compared to the rate of protein breakdown, leading to the onset of rapid muscle degeneration. This may expose individuals to muscle weakness-related diseases.

Sarcopenia, which is one muscle weakness-related disease, is a condition in which approximately 13 to 24% of the usual body mass is lost, indicating decreases in protein content, fiber diameter, muscle force production, and fatigue resistance. Sarcopenia is caused by a variety of factors, including sepsis, cancer, renal failure, glucocorticoid excess, denervation, muscle disuse, obesity, and aging. The main cause of sarcopenia may be considered to be the gradual decrease in quantity and quality of skeletal muscle that occurs with aging.

Accordingly, research and efforts are being focused on treating muscle loss caused by common muscle weakness-related diseases or increasing muscle mass. Therefore, there is still a need for research on the treatment of muscle weakness-related diseases and muscle strengthening.

Meanwhile, FGF18 is a member of the fibroblast growth factor (FGF) family. The FGF18 protein is known to induce neurite outgrowth in PC12 cells in vitro, and FGF18 is known to bind to a specific receptor (Fibroblast Growth Factor Receptor, FGFR) and activate intracellular signaling pathways.

However, there is currently no treatment for sarcopenia, and it is not yet known whether FGF18 is effective in preventing, treating, or diagnosing muscle weakness-related diseases. The present inventors have confirmed that blocking the FGF18 signaling pathway that causes muscle weakness-related diseases is effective in preventing or treating the diseases. Also, the present inventors have discovered that muscle weakness-related diseases occur when FGF18 is treated. Therefore, the present invention has been completed this finding.

### PRIOR ART DOCUMENTS

### Patent Documents

(Patent Document 1) Korean Patent Application No. 10-2020-0034910.

### DISCLOSURE

### Technical Problem

The present invention aims to provide a method of screening for a prophylactic or therapeutic agent for a muscle weakness-related disease, comprising:
(a) treating cells expressing a fibroblast growth factor 18 (FGF18) protein or an mRNA thereof with a test material;
(b) measuring an expression level of the FGF18 protein or mRNA thereof in the cells treated with the test material and the cells not treated with the test material; and
(c) screening for a material that reduces the expression level of the FGF18 protein or mRNA thereof compared to control cells as a prophylactic or therapeutic agent for the muscle weakness-related disease.

Another object of the present invention is to provide a method of screening for a prophylactic or therapeutic agent for a muscle weakness-related disease, comprising:
(a) treating cells expressing a fibroblast growth factor 18 (FGF18) protein or an mRNA thereof with a test material;
(b) measuring an activity level of a fibroblast growth factor receptor 4 (FGFR4)-mediated FGF18 signaling pathway; and
(c) screening for a material that inhibits the activity of the FGFR4-mediated FGF18 signaling pathway as a prophylactic or therapeutic agent for the muscle weakness-related disease.

Another object of the present invention is to provide a pharmaceutical composition for preventing or treating a muscle weakness-related disease, comprising, as an active ingredient, an agent for inhibiting the expression of a fibroblast growth factor receptor 4 (FGFR4) gene or the activity of the protein.

Still another object of the present invention is to provide a method of characterizing a responder to a therapeutic agent for a muscle weakness-related disease to treat a muscle weakness-related disease, the method comprising:
(S1) measuring an expression level of a fibroblast growth factor 18 (FGF18) protein or an mRNA thereof in a biological sample isolated from a subject; and
(S2) determining a responder to the therapeutic agent for a muscle weakness-related disease when the expression level of the FGF18 protein or mRNA thereof measured in the step (S1) increases compared to a control,
wherein the therapeutic agent for a muscle weakness-related disease is the composition of the present invention.

Another object of the present invention is to provide a composition for diagnosing a muscle weakness-related disease, comprising, as an active ingredient, an agent for measuring an expression level of a fibroblast growth factor 18 (FGF18) protein or an mRNA of a gene encoding the protein.

Another object of the present invention is to provide a kit for diagnosing a muscle weakness-related disease, comprising an agent for measuring a level of a fibroblast growth factor 18 (FGF18) protein or an mRNA of a gene encoding the protein.

Another object of the present invention is to provide a method for providing information on diagnosing a muscle weakness-related disease, comprising: measuring an expression level of a fibroblast growth factor 18 (FGF18) protein or an mRNA of a gene encoding the protein in a biological sample isolated from a subject.

However, technical problems to be solved in the present invention are not limited to the above-described problems, and other problems which are not described herein will be fully understood by those of ordinary skill in the art from the following descriptions.

### Technical Solution

To achieve the above objects, the present invention provides a method of screening for a prophylactic or therapeutic agent for a muscle weakness-related disease, comprising:
(a) treating cells expressing a fibroblast growth factor 18 (FGF18) protein or an mRNA thereof with a test material;
(b) measuring an expression level of the FGF18 protein or mRNA thereof in the cells treated with the test material and the cells not treated with the test material; and
(c) screening for a material that reduces the expression level of the FGF18 protein or mRNA thereof compared to control cells as a prophylactic or therapeutic agent for the muscle weakness-related disease.

The present invention provides a method of screening for a prophylactic or therapeutic agent for a muscle weakness-related disease, comprising:
(a) treating cells expressing a fibroblast growth factor 18 (FGF18) protein or an mRNA thereof with a test material;
(b) measuring an activity level of a fibroblast growth factor receptor 4 (FGFR4)-mediated FGF18 signaling pathway; and
(c) screening for a material that inhibits the activity of the FGFR4-mediated FGF18 signaling pathway as a prophylactic or therapeutic agent for the muscle weakness-related disease.

In one embodiment of the present invention, the muscle weakness-related disease may be one or more selected from the group consisting of sarcopenia, muscle atrophy, muscle dystrophy, and cardiac atrophy, but is not limited thereto.

In another embodiment of the present invention, the cells expressing the FGF18 protein or mRNA thereof may be isolated from a patient with a muscle weakness-related disease, but are not limited thereto.

The present invention provides a composition for screening a prophylactic or therapeutic agent for a muscle weakness-related disease, comprising, as an active ingredient, an agent for measuring an expression level of a fibroblast growth factor 18 (FGF18) protein or an mRNA of a gene encoding the protein.

The present invention provides a composition for screening a prophylactic or therapeutic agent for a muscle weakness-related disease, comprising, as an active ingredient, an agent for measuring an activity level of a fibroblast growth factor receptor 4 (FGFR4)-mediated FGF18 signaling pathway.

The present invention provides a kit for screening a prophylactic or therapeutic agent for a muscle weakness-related disease, comprising the screening composition of the present invention and an instruction manual.

The present invention provides a pharmaceutical composition for preventing or treating a muscle weakness-related disease, comprising, as an active ingredient, an agent for inhibiting the expression of a fibroblast growth factor receptor 4 (FGFR4) gene or the activity of the protein.

In one embodiment of the present invention, the agent binds to FGFR4 to inhibit an FGF18 signaling pathway mediated by the FGFR4, but is not limited thereto.

In another embodiment of the present invention, the muscle weakness-related disease may be one or more selected from the group consisting of sarcopenia, muscle atrophy, muscle dystrophy, and cardiac atrophy, but is not limited thereto.

In yet another embodiment of the present invention, the composition may satisfy one or more characteristics selected from the group consisting of:
(a) reducing the proliferation of satellite cells;
(b) increasing the diameters of myotube cells;
(c) increasing skeletal muscle mass; and
(d) increasing one or more exercise abilities selected from the group consisting of muscle strength, muscle endurance, and maximal speed, but is not limited thereto.

In yet another embodiment of the present invention, the agent for inhibiting the expression of the gene may be one or more selected from the group consisting of miRNA, siRNA, shRNA, a ribozyme, a DNAzyme, peptide nucleic acid (PNA), and an antisense oligonucleotide which complementarily bind to the mRNA of the FGFR4 gene, and
the agent for inhibiting the activity of the protein may be one or more selected from the group consisting of a peptide, an antibody, an aptamer, and a compound which specifically bind to the FGFR4 protein, but is not limited thereto.

The present invention provides a kit for preventing or treating a muscle weakness-related disease, comprising the pharmaceutical composition for preventing or treating a muscle weakness-related disease of the present invention and an instruction manual.

The present invention provides a method of characterizing a responder to a therapeutic agent for a muscle weakness-related disease to treat a muscle weakness-related disease, the method comprising:
(S1) measuring an expression level of a fibroblast growth factor 18 (FGF18) protein or an mRNA thereof in a biological sample isolated from a subject; and
(S2) determining a responder to the therapeutic agent for a muscle weakness-related disease when the expression level of the FGF18 protein or mRNA thereof measured in the step (S1) increases compared to a control,
wherein the therapeutic agent for a muscle weakness-related disease is the composition of the present invention.

In one embodiment of the present invention, the muscle weakness-related disease may be one or more selected from the group consisting of sarcopenia, muscle atrophy, muscle dystrophy, and cardiac atrophy, but is not limited thereto.

The present invention provides a composition for characterizing a responder to a therapeutic agent for a muscle weakness-related disease, comprising, as an active ingredient, an agent for measuring an expression level of a fibroblast growth factor 18 (FGF18) protein or an mRNA of a gene encoding the protein.

The present invention provides a kit for characterizing a responder to a therapeutic agent for a muscle weakness-related disease, comprising the composition for characterizing a responder to a therapeutic agent for a muscle weakness-related disease of the present invention and an instruction manual.

The present invention provides a composition for diagnosing a muscle weakness-related disease, comprising, as an active ingredient, an agent for measuring an expression level of a fibroblast growth factor 18 (FGF18) protein or an mRNA of a gene encoding the protein.

In one embodiment of the present invention, the muscle weakness-related disease may be one or more selected from the group consisting of sarcopenia, muscle atrophy, muscle dystrophy, and cardiac atrophy, but is not limited thereto.

In another embodiment of the present invention, the muscle weakness-related disease satisfies one or more characteristics selected from the group consisting of:
(a) decreased skeletal muscle mass;
(b) decreased exercise ability selected from the group consisting of muscle strength, muscle endurance, and maximal speed;
(c) inhibited muscle regeneration or growth;
(d) decreased diameters of myotube cells;
(e) increased activity or proliferation of satellite cells; and
(f) decreased differentiation of satellite cells, but is not limited thereto.

In yet another embodiment of the present invention, the agent for measuring the protein expression level may be an antibody specific for an FGF18 protein, but is not limited thereto.

In yet another embodiment of the present invention, the agent for measuring the mRNA expression level may be a probe or a primer set that specifically binds to the mRNA of FGF18, but is not limited thereto.

The present invention provides a kit for diagnosing a muscle weakness-related disease, comprising an agent for measuring a level of a fibroblast growth factor 18 (FGF18) protein or an mRNA of a gene encoding the protein.

In one embodiment of the present invention, the kit may further comprise an instruction manual, but is not limited thereto.

The present invention provides a method for providing information on diagnosing a muscle weakness-related disease, comprising: measuring an expression level of a fibroblast growth factor 18 (FGF18) protein or an mRNA of a gene encoding the protein in a biological sample isolated from a subject.

In one embodiment of the present invention, the method for providing information on diagnosing a muscle weakness-related disease may further comprise determining that a subject having a decreased level of one or more selected from the group consisting of the following, as compared to a normal control sample, is suffering from a muscle weakness-related disease, but is not limited thereto:
(S1) an expression level of a protein associated with sarcomere formation and muscle contraction, or an mRNA of a gene encoding the protein, in a biological sample isolated from the subject;
(S2) an expression level of a downstream protein associated with actin filaments responsible for sarcomere formation and movement, or filament-based movements, or an mRNA of a gene encoding the protein, in a biological sample isolated from the subject; and
(S3) an expression level of a protein associated with a cytoskeleton signaling pathway in muscle cells, or an mRNA of a gene encoding the protein, in a biological sample isolated from the subject.

In another embodiment of the present invention, the method may comprise comparing the expression level of the FGF18 protein or the mRNA of the gene encoding the protein with a normal control sample to determine that a subject having an increased expression level is suffering from a muscle weakness-related disease, but is not limited thereto.

In yet another embodiment of the present invention, the muscle weakness-related disease may be one or more selected from the group consisting of sarcopenia, muscle atrophy, muscle dystrophy, and cardiac atrophy, but is not limited thereto.

In yet another embodiment of the present invention, the biological sample may be any one or more selected from the group consisting of tissue, cells, whole blood, blood, serum, and plasma, but is not limited thereto.

In yet another embodiment of the present invention, the protein expression level may be measured using one or more methods selected from the group consisting of western blot, ELISA (enzyme linked immunoassay), radioimmunoassay, radioimmunodiffusion, Ouchterlony immunodiffusion, rocket immunoelectrophoresis, immunohistochemistry, immunoprecipitation assay, complement fixation assay, fluorescence-activated cell sorting (FACS), and protein chips, but is not limited thereto.

In yet another embodiment of the present invention, the mRNA expression level may be measured using one or more methods selected from the group consisting of reverse transcription polymerase chain reaction (RT-PCR), competitive RT-PCR, real-time RT-PCR, quantitative or semi-quantitative RT-PCR, quantitative or semi-quantitative real-time RT-PCR, in situ hybridization, fluorescence in situ hybridization (FISH), RNase protection assay (RPA), northern blotting, Southern blotting, RNA sequencing, DNA chips, and RNA chips, but is not limited thereto.

In yet another embodiment of the present invention, the kit may further comprise an instruction manual that teaches the content including the above method, but is not limited thereto.

Additionally, the present invention provides a method for preventing or treating a muscle weakness-related disease, comprising administering to a subject in need thereof a pharmaceutically effective amount of a composition comprising, as an active ingredient, an agent for inhibiting the expression of a fibroblast growth factor receptor 4 (FGFR4) gene or the activity of the protein.

Additionally, the present invention provides a use of a composition comprising, as an active ingredient, an agent for inhibiting the expression of a fibroblast growth factor receptor 4 (FGFR4) gene or the activity of the protein for preventing or treating a muscle weakness-related disease.

Additionally, the present invention provides a use of a composition comprising, as an active ingredient, an agent for inhibiting the expression of a fibroblast growth factor receptor 4 (FGFR4) gene or the activity of the protein for preparing a medicament for preventing or treating a muscle weakness-related disease.

Additionally, the present invention provides a use of an agent for measuring an expression level of a fibroblast growth factor 18 (FGF18) protein or an mRNA of a gene encoding the protein, or a composition comprising the agent as an active ingredient, for diagnosing a muscle weakness-related disease; for screening a prophylactic or therapeutic agent for a muscle weakness-related disease; or for characterizing a responder to a therapeutic agent for a muscle weakness-related disease.

Additionally, the present invention provides a use of an agent for measuring an expression level of a fibroblast growth factor 18 (FGF18) protein or an mRNA of a gene encoding the protein, or a composition comprising the agent as an active ingredient, for preparing a medicament for diagnosing a muscle weakness-related disease; for screening a prophylactic or therapeutic agent for a muscle weakness-related disease; or for characterizing a responder to a therapeutic agent for a muscle weakness-related disease.

Additionally, the present invention provides a method for diagnosing a muscle weakness-related disease, comprising:
(S1) measuring an expression level of a fibroblast growth factor 18 (FGF18) protein or an mRNA of a gene encoding the protein in a biological sample isolated from a subject; and
(S2) determining that a subject having an increased expression level of the FGF18 protein or an mRNA of a gene encoding the protein, as compared to a normal control sample, is suffering from a muscle weakness-related disease.

Additionally, the present invention provides a method for treating a muscle weakness-related disease, comprising:
(S1) measuring an expression level of a fibroblast growth factor 18 (FGF18) protein or an mRNA of a gene encoding the protein in a biological sample isolated from a subject;
(S2) determining that a subject having an increased expression level of the FGF18 protein or an mRNA of a gene encoding the protein, as compared to a normal control sample, is suffering from a muscle weakness-related disease; and
(S3) treating the subject suffering from the muscle weakness-related disease.

### Advantageous Effects

The present invention utilizes the FGFR4-mediated FGF18 signaling pathway that causes muscle weakness-related diseases to diagnose the muscle weakness-related diseases, to screen for a prophylactic or therapeutic agent for the diseases, and to prevent or treat the muscle weakness-related diseases by blocking the signaling pathway. Additionally, the responder characterization method of the present invention may effectively exhibit therapeutic effects for muscle weakness-related diseases by preferentially determining whether a subject is a responder to the therapeutic agent of the present invention before introducing a treatment method. Specifically, when the level of FGF18 associated with the FGFR4-mediated FGF18 signaling pathway increased, symptoms caused by muscle weakness-related diseases, such as decreased skeletal muscle mass; decreased exercise abilities such as muscle strength, muscle endurance, and maximal speed; inhibited muscle regeneration and growth; decreased diameters of myotube cells; and increased satellite cell activity, increased proliferation, and decreased differentiation, were observed. Also, when an FGFR4 inhibitor associated with the FGFR4-mediated FGF18 signaling pathway was administered, it was confirmed that the symptoms caused by the muscle weakness-related diseases were alleviated. Therefore, the FGFR4-mediated FGF18 signaling pathway of the present invention may be utilized to diagnose, treat, and prevent muscle weakness-related diseases.

### DESCRIPTION OF DRAWINGS

FIG. 1A illustrates the sequences of FGF18, FGF2, and FGF21.

FIG. 1B illustrates the results confirming changes in skeletal muscle mass in each group administered with FGF18, FGF2, or FGF21 after muscle damage induced by CTX.

FIG. 1C illustrates the results confirming changes in muscle strength in each group administered with FGF18, FGF2, or FGF21 after muscle damage induced by CTX.

FIG. 1D illustrates the results confirming changes in muscle endurance in each group administered with FGF18, FGF2, or FGF21 after muscle damage induced by CTX.

FIG. 1E illustrates the results confirming changes in maximal speed in each group administered with FGF18, FGF2, or FGF21 after muscle damage induced by CTX.

FIG. 1F illustrates the results confirming the degree of inhibition of muscle regeneration and growth in each group administered with FGF18, FGF2, or FGF21 after muscle damage induced by CTX.

FIG. 2A illustrates the results confirming the degree of satellite cell proliferation in each group administered with FGF18, FGF2, or FGF21.

FIG. 2B illustrates the results confirming changes in the diameters of myotube cells in each group administered with FGF18 or FGF21.

FIG. 2C illustrates the results measuring the proportions of PAX7- and tdTomato-positive satellite cells in the group administered with FGF18.

FIG. 2D illustrates the results measuring the proportions of MyoG- and tdTomato-positive satellite cells in the group administered with FGF18.

FIG. 2E illustrates the results measuring the proportions of PAX7-, MyoD-, or MyoG-positive satellite cells in each group administered with FGF18 or FGF21.

FIG. 3A illustrates the results showing decreased skeletal muscle exercise ability in a liver disease mouse model.

FIG. 3B illustrates the results showing that, in a liver disease mouse model, the expression of PAX7, a marker of satellite cells, and MyoD, an activation marker of satellite cells, increased, whereas the expression of MyoG, a differentiation marker of satellite cells, did not change.

FIG. 3C illustrates the results showing increased intramuscular cell proliferation in a liver disease mouse model.

FIG. 3D illustrates the results showing increased proliferation of skeletal muscle satellite cells in a liver disease mouse model.

FIG. 3E illustrates the results showing decreased muscle strength in a liver disease mouse model.

FIG. 3F illustrates the results showing decreased muscle endurance in a liver disease mouse model.

FIG. 3G illustrates the results showing decreased maximal speed in a liver disease mouse model.

FIG. 3H illustrates the results showing decreased skeletal muscle regeneration ability in a liver disease mouse model after muscle damage induced by CTX.

FIG. 4A illustrates the results showing increased hepatic Fgf18 mRNA levels in a liver disease mouse model.

FIG. 4B illustrates the results showing that hepatic Fgf18 mRNA expression inversely correlates with skeletal muscle exercise ability in a liver disease mouse model.

FIG. 4C illustrates the results showing increased hepatic FGF18 protein levels in a liver disease mouse model.

FIG. 4D illustrates the results showing increased circulating FGF18 protein levels in the blood of a liver disease mouse model.

FIG. 4E illustrates the results showing that circulating FGF18 protein levels in the blood of a liver disease mouse model inversely correlate with skeletal muscle exercise ability.

FIG. 4F illustrates the results showing increased hepatic Fgf18 mRNA levels in aging and liver disease mouse models.

FIG. 4G illustrates the results showing increased circulating FGF18 protein levels in the blood of an aging mouse model.

FIG. 4H illustrates the primer sequences used for performing real-time PCR.

FIG. 5A illustrates the results showing increased expression of FGFR4, a receptor for FGF18, in satellite cells of a liver disease mouse model.

FIG. 5B illustrates the results showing that FGF18-induced satellite cell proliferation decreased when co-treated with an FGFR4 inhibitor.

FIG. 5C illustrates the results showing that the diameter of myotube cells increased when an FGFR4 inhibitor was administered to myotube cells treated with FGF18.

FIGS. 6A to 6E illustrate the results showing decreased expression of genes associated with skeletal muscle cytostructure formation in satellite cells treated with FGF18.

FIG. 7A illustrates the results confirming changes in skeletal muscle mass when an FGFR4 inhibitor was administered to the group treated with FGF18 after muscle damage induced by CTX.

FIG. 7B illustrates the results confirming changes in muscle strength when an FGFR4 inhibitor was administered to the group treated with FGF18 after muscle damage induced by CTX.

FIG. 7C illustrates the results confirming changes in muscle endurance when an FGFR4 inhibitor was administered to the group treated with FGF18 after muscle damage induced by CTX.

FIG. 7D illustrates the results confirming changes in maximal speed when an FGFR4 inhibitor was administered to the group treated with FGF18 after muscle damage induced by CTX.

### BEST MODE

The present invention relates to a method for preventing or treating a muscle weakness-related disease by inhibiting the activity of the FGFR4-mediated FGF18 signaling pathway in satellite cells of skeletal muscle, thereby suppressing muscle loss and atrophy. The present invention also relates to a method for diagnosing a muscle weakness-related disease by measuring a level of FGF18.

The present invention provides a method of screening for a prophylactic or therapeutic agent for a muscle weakness-related disease, comprising:
(a) treating cells expressing a fibroblast growth factor 18 (FGF18) protein or an mRNA thereof with a test material;
(b) measuring an expression level of the FGF18 protein or mRNA thereof in the cells treated with the test material and the cells not treated with the test material; and
(c) screening for a material that reduces the expression level of the FGF18 protein or mRNA thereof compared to control cells as a prophylactic or therapeutic agent for the muscle weakness-related disease.

The present invention provides a method of screening for a prophylactic or therapeutic agent for a muscle weakness-related disease, comprising:
(a) treating cells expressing a fibroblast growth factor 18 (FGF18) protein or an mRNA thereof with a test material;
(b) measuring an activity level of a fibroblast growth factor receptor 4 (FGFR4)-mediated FGF18 signaling pathway; and
(c) screening for a material that inhibits the activity of the FGFR4-mediated FGF18 signaling pathway as a prophylactic or therapeutic agent for the muscle weakness-related disease.

In one embodiment of the present invention, the muscle weakness-related disease is one or more selected from the group consisting of sarcopenia, muscle atrophy, muscle dystrophy, and cardiac atrophy, but is not limited thereto.

In the present invention, “fibroblast growth factor 18 (FGF18)” is a growth factor mainly secreted from the liver, and its expression may increase in metabolic diseases such as aging and metabolic associated steatohepatitis (MASH). Circulating FGF18 in the body may act on fibroblast growth factor receptors (FGFRs) present in skeletal muscle satellite cells to activate a signaling pathway that induces muscle loss in muscle tissue, and such an action may cause atrophic muscle degeneration and ultimately induce sarcopenia, but is not limited thereto. Additionally, in the present invention, FGF18 may refer to an FGF18 protein or an FGF18 gene, but is not limited thereto.

In the present invention, “FGF18” is not particularly limited in its specific origin or sequence (amino acid sequence constitution) as long as it is known in the art as an FGF18 protein or polypeptide, but may include, for example, an amino acid sequence represented by Sequence ID No. 1 as a human (Homo sapiens)-derived FGF18 in the present invention, and may preferably be a polypeptide consisting of the amino acid sequence of Sequence ID No. 1, but is not limited thereto. Additionally, FGF18 may be encoded by the nucleotide sequences of NCBI Reference Sequence: AB528345.1, BT019570.1, or AB007422.1, but is not limited thereto.

In the present invention, FGF18, FGF2, and FGF21 may each be used interchangeably with recombinant proteins FGF18, FGF2, and FGF21, respectively, but are not limited thereto.

In the present invention, a “muscle weakness-related disease” means any disease in which muscle tissue or muscle cells decrease or are lost. The muscle weakness may be localized to a particular muscle, one side of the body, the upper limbs or the lower limbs, may appear throughout the entire body, and may occur congenitally or acquiredly. Additionally, subjective symptoms of muscle weakness, including muscle fatigue or myalgia, may be objectively quantified through physical examinations. The muscle weakness-related disease may include, for example, sarcopenia, muscle atrophy, muscle dystrophy, and cardiac atrophy, but is not limited thereto, and includes all diseases caused by a decrease or loss of muscle tissue or muscle cells.

In the present invention, “sarcopenia” refers to a condition in which the muscles of the body (muscle mass and muscle strength) abnormally decrease or weaken due to various reasons such as aging or obesity, resulting in impaired physical activity, and when the symptoms worsen, it leads to disability and increases the risk of death. Sarcopenia affects not only the muscle itself but also the entire body, including bones, blood vessels, nerves, the liver, the heart, and the pancreas. Additionally, it is known that when muscle decreases, it interferes with the formation of new blood vessels and nerves, thereby increasing the risk of cognitive decline, fatty liver, and diabetes.

Additionally, in the present invention, “sarcopenia” may be aging-associated sarcopenia, obesity-associated sarcopenia, or sarcopenia caused by a metabolic disease, and may be sarcopenia caused by one or more selected from the group consisting of aging, obesity, steatohepatitis, chronic liver disease, metabolic associated steatohepatitis (MASH), metabolic syndrome, hyperinsulinemia, hyperlipidemia, hypertriglyceridemia, hypercholesterolemia, dyslipidemia, fatty liver, and arteriosclerosis, but is not limited thereto.

In the present invention, “aging-associated sarcopenia” refers to a condition in which muscle mass gradually decreases or the density and function of muscle gradually weaken due to aging, directly causing a decline in muscle strength and consequently leading to reduced physical functions and disability.

In the present invention, “obesity-associated sarcopenia” refers to a condition in which fat is deposited in muscle due to obesity, leading to a decrease in muscle mass, an increase in obesity cell-derived inflammatory factors, and a decline in mitochondrial function in muscle, thereby weakening muscle function, and when insulin secretion becomes abnormal due to obesity, energy cannot be properly supplied to cells, which may cause impaired muscle development.

In the present invention, “obesity” refers to a state in which excess energy accumulates in the body due to an imbalance between energy intake and consumption, resulting in an abnormal increase in adipose tissue, and even if one appears to have a normal body weight, one may be considered obese if the body fat percentage is high. Obesity arises not from a single cause but from multiple causes acting in combination, and may result from improper dietary habits including westernized eating patterns, reduced physical activity, emotional factors, and genetic factors. In the present invention, the obesity may be sarcopenic obesity, but is not limited thereto.

In the present invention, a “metabolic disease” refers to a condition or disease that is closely associated with obesity or caused by obesity, and may be, for example, dyslipidemia; hepatotoxic diseases including drug-induced liver injury, viral liver injury, hepatitis, liver cirrhosis, liver cancer, or hepatic encephalopathy; or fatty liver, but is not limited thereto.

In the present invention, “muscle atrophy” refers to a condition in which the muscles of the limbs gradually atrophy, and may cause progressive degeneration of motor nerve fibers and cells in the spinal cord, leading to amyotrophic lateral sclerosis (ALS) and spinal progressive muscular atrophy (SPMA).

In the present invention, “muscle dystrophy” refers to a disease in which progressive muscle atrophy and muscle weakness occur, and pathologically refers to a degenerative myopathy characterized by necrosis of muscle fibers.

In the present invention, the “cardiac atrophy” refers to a condition in which the heart becomes atrophic due to external or internal factors, and manifests as symptoms in which myocardial fibers become thin and wasted by starvation, wasting diseases, or senility, leading to a reduction in adipose tissue.

In the present invention, the liver disease may be one or more selected from the group consisting of aging, metabolic associated steatohepatitis, and liver cancer.

In the present invention, “metabolic dysfunction-associated steatotic liver disease (MASLD)” may be used interchangeably with nonalcoholic fatty liver disease (NAFLD). Metabolic dysfunction-associated steatotic liver disease is a type of fatty liver disease that occurs when fat accumulates in the liver of a patient who does not consume excessive alcohol, and refers to a broad spectrum of diseases including simple steatosis without inflammatory responses and advanced stages thereof involving hepatocellular inflammation, hepatic fibrosis, and liver cirrhosis. Metabolic dysfunction-associated steatotic liver disease may yield good outcomes when detected at an early stage, but if not, it may progress to metabolic associated steatohepatitis (MASH) due to various reasons, and further develop into liver cirrhosis and liver cancer.

In the present invention, “metabolic associated steatohepatitis (MASH)” may be used interchangeably with nonalcoholic steatohepatitis (NASH).

In one embodiment of the present invention, the cell expressing a protein of FGF18 or an mRNA thereof may be isolated from a subject suffering from a muscle weakness-related disease, but is not limited thereto. Additionally, in the present invention, the “cell expressing a protein of FGF18 or an mRNA thereof” may be replaced with a “biological sample isolated from a subject suffering from a muscle weakness-related disease,” but is not limited thereto.

In the present invention, the “biological sample” may be any one or more selected from the group consisting of blood, serum, whole blood, plasma, tissue, cells, satellite cells, myotube cells, tibialis anterior muscle, organs, bone marrow, fine needle aspiration samples, core needle biopsy samples, and vacuum-assisted biopsy samples, but is not limited thereto.

In the present invention, the “test material” means an unknown substance used in screening to examine whether it affects the expression level of the FGF18 protein or an mRNA thereof in a cell (or biological sample) expressing the FGF18 protein or an mRNA thereof. The test material may include siRNA (small interference RNA), shRNA (short hairpin RNA), miRNA (microRNA), ribozyme, DNAzyme, PNA (peptide nucleic acids), antisense oligonucleotides, a recombinant plasmid, nanoparticles, proteins, oligopeptides, antibodies, aptamers, natural extracts, or chemicals, but is not limited thereto. In the present invention, the “test material” may be used interchangeably with a candidate prophylactic or therapeutic agent for a muscle weakness-related disease, but is not limited thereto.

In the present invention, treating with a test material may mean adding the test material to a culture medium of a cell or biological sample and then culturing the cell or biological sample for a certain period of time, and may mean contacting the test material therewith, but is not limited thereto. When the biological sample is provided in the form of a laboratory animal, the contact with the test material is not limited to non-oral or oral administration or stereotaxic injection, and one skilled in the art may select an appropriate method to test the test material in the animal.

In the present invention, the term “control group,” as used in reference to the screening method, may refer to cells (biological samples) that are not treated with a test material, but is not limited thereto.

In the present invention, the measurement of the expression level of an FGF18 protein may be performed using one or more methods selected from the group consisting of western blotting, ELISA (enzyme linked immunoassay), radioimmunoassay, radioimmunodiffusion, Ouchterlony immunodiffusion, rocket immunoelectrophoresis, immunohistochemical staining, immunoprecipitation assay, complement fixation assay, fluorescence-activated cell sorting (FACS), and protein chips.

In the present invention, the measurement of the expression level of the mRNA thereof may be performed using one or more methods selected from the group consisting of reverse transcription polymerase chain reaction (RT-PCR), competitive RT-PCR, real-time RT-PCR, quantitative or semi-quantitative RT-PCR, quantitative or semi-quantitative real-time RT-PCR, in situ hybridization, fluorescence in situ hybridization (FISH), RNase protection assay (RPA), northern blotting, Southern blotting, RNA sequencing, DNA chips, and RNA chips, but is not limited thereto.

In the present invention, “selecting” may be used interchangeably with distinguishing, determining, or judging, but is not limited thereto.

In one embodiment of the present invention, “measuring the activity level of the FGFR4-mediated FGF18 signaling pathway” may be performed through one or more selected from the group consisting of satellite cell proliferation, myotube cell diameter, skeletal muscle mass, and physical performance.

Specifically, when the “activity of the FGFR4-mediated FGF18 signaling pathway is inhibited,” the increase in satellite cell proliferation or the decrease in myotube cell diameter observed upon treating satellite cells with FGF18 may be reversed, such that satellite cell proliferation decreases or myotube cell diameter increases when an FGFR4 inhibitor is co-administered. Additionally, when the “activity of the FGFR4-mediated FGF18 signaling pathway is inhibited,” the decrease in skeletal muscle mass or physical performance observed upon treating an in vivo mouse model with FGF18 may be reversed, such that skeletal muscle mass increases or physical performance improves when an FGFR4 inhibitor is co-administered.

The present invention provides a pharmaceutical composition for preventing or treating a muscle weakness-related disease, comprising, as an active ingredient, an agent for inhibiting the expression of a fibroblast growth factor receptor 4 (FGFR4) gene or the activity of the protein.

In the present invention, fibroblast growth factor receptor 4 (FGFR4) is a tyrosine kinase and a cell surface receptor for fibroblast growth factors, and the encoded protein comprises an extracellular region consisting of three immunoglobulin-like domains, a single hydrophobic transmembrane segment, and a cytoplasmic tyrosine kinase domain, and the extracellular portion may interact with fibroblast growth factors to induce a cascade of downstream signaling, but is not limited thereto. Additionally, in the present invention, FGFR4 may refer to an FGFR4 protein or an FGFR4 gene, but is not limited thereto.

In the present invention, “FGFR4” is not particularly limited in its specific origin or sequence (amino acid sequence constitution) as long as it is known in the art as an FGFR4 protein or polypeptide, but may include, for example, an amino acid sequence represented by NCBI Reference Sequence: NP_001278909.1 as a human (Homo sapiens)-derived FGFR4 in the present invention, and may preferably be a polypeptide consisting of the amino acid sequence, but is not limited thereto. Additionally, FGFR4 may be encoded by the nucleotide sequence of NCBI Gene ID: 2264, but is not limited thereto.

In one embodiment of the present invention, the agent may bind to FGFR4 to inhibit the FGFR4-mediated FGF18 signaling pathway, but is not limited thereto.

In the present invention, FGF18 increases in the liver under metabolic disease or aging conditions such as metabolic associated steatohepatitis (MASH), circulates in the body, and reaches skeletal muscle, and when the level of FGF18 in the body increases, it may cause a decrease in skeletal muscle mass and muscle function. Additionally, skeletal muscle satellite cells, which are adult stem cells responsible for muscle damage and repair, express FGFR4, and under conditions such as aging or metabolic associated steatohepatitis (MASH), the expression of FGFR4 in skeletal muscle satellite cells may increase. FGFR4 is a receptor for FGF18, and increased expression of FGFR4 may activate the FGFR4-mediated FGF18 signaling pathway, thereby decreasing the expression of genes related to sarcomere and actin formation involved in skeletal muscle structure.

To date, the development of therapeutics for sarcopenia has been directed toward targeting the cells and structure of the muscle itself, and the development of sarcopenia therapeutics from a metabolic perspective, such as targeting the FGF18 signaling pathway, has not been pursued. Therefore, the therapeutic effect on muscle weakness-related diseases through inhibition of the FGF18 signaling pathway according to the present invention, specifically the therapeutic effect on sarcopenia, may advance into the development of new therapeutics at a time when no approved drug is currently available. Additionally, it may be widely used not only for treating sarcopenia but also for prevention and improvement thereof, and as a substance capable of replacing excessive steroid misuse for muscle hypertrophy.

In one embodiment of the present invention, “inhibiting the FGFR4-mediated FGF18 signaling pathway” may be confirmed through one or more selected from the group consisting of satellite cell proliferation, myotube cell diameter, skeletal muscle mass, and physical performance.

Specifically, when the “FGFR4-mediated FGF18 signaling pathway is inhibited,” the increase in satellite cell proliferation or the decrease in myotube cell diameter observed upon treating satellite cells with FGF18 may be reversed, such that satellite cell proliferation decreases or myotube cell diameter increases when an FGFR4 inhibitor is co-administered. Additionally, when the “FGFR4-mediated FGF18 signaling pathway is inhibited,” the decrease in skeletal muscle mass or physical performance observed upon treating an in vivo mouse model with FGF18 may be reversed, such that skeletal muscle mass increases or physical performance improves when an FGFR4 inhibitor is co-administered.

In one embodiment of the present invention, the muscle weakness-related disease may be one or more selected from the group consisting of sarcopenia, muscle atrophy, muscle dystrophy, and cardiac atrophy, but is not limited thereto.

In one embodiment of the present invention, the composition may satisfy one or more characteristics selected from the group consisting of:
(a) reducing the proliferation of satellite cells;
(b) increasing the diameters of myotube cells;
(c) increasing skeletal muscle mass; and
(d) increasing one or more exercise abilities selected from the group consisting of muscle strength, muscle endurance, and maximal speed.

In the present invention, in “(a) reducing the proliferation of satellite cells,” the decrease in satellite cell proliferation may mean a decrease in KI67, which is a marker of proliferating cells, but is not limited thereto.

In the present invention, in “(b) increasing the diameters of myotube cells,” the increase in myotube cell diameter may be confirmed through MyHC, but is not limited thereto.

In the present invention, in “(c) increasing skeletal muscle mass,” the skeletal muscle may be one or more selected from the group consisting of the quadriceps (Quad), gastrocnemius (GAS), soleus (Sol), tibialis anterior (TA), and extensor digitorum longus (EDL), but is not limited thereto.

In the present invention, in “(d) increasing one or more exercise abilities selected from the group consisting of muscle strength, muscle endurance, and maximal speed,” muscle strength may be measured using a grip strength meter, muscle endurance may be measured through a Grid-Hanging test, and maximal speed may be measured through a treadmill test, but is not limited thereto.

In one embodiment of the present invention, the agent inhibiting the expression of the FGFR4 gene is one or more selected from the group consisting of miRNA, siRNA, shRNA, ribozyme, DNAzyme, PNA (peptide nucleic acid), and antisense oligonucleotides that complementarily bind to an mRNA of the FGFR4 gene, and the agent inhibiting the activity of the FGFR4 protein may be one or more selected from the group consisting of peptides, antibodies, aptamers, and compounds that specifically bind to the FGFR4 protein, but is not limited thereto.

In one embodiment of the present invention, the agent inhibiting the expression of the FGFR4 gene or the activity of the FGFR4 protein may be BLU9931 (C26H22C12N4O3), but is not limited thereto.

In the present invention, BLU9931 may be a compound represented by Chemical Formula 1 below, but is not limited thereto. Additionally, BLU9931 may refer to BLU9931 in its ordinary meaning, and may be a compound synthesized by one skilled in the art or commercially available, but is not limited thereto.

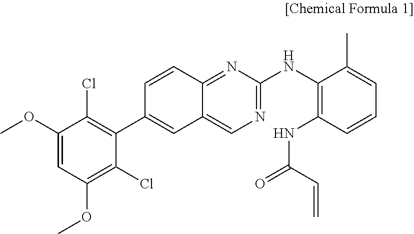 [SMILES: C=CC(=O)Nc1cccc(C)c1Nc1ncc2cc(-c3c(Cl)c(OC)cc(OC)c3Cl)ccc2n1]

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO3) carbon dioxide gas, sodium metabisulfite (Na2S2O5), sodium sulfite (Na2SO3), nitrogen gas (N2), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan (propylene glycol monostearate), glycerin, Adeps solidus, buytyrum Tego-G, cebes Pharma 16, hexalide base 95, cotomar, Hydrokote SP, S-70-XXA, S-70-XX75 (S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As used herein, the “patient” refers to a subject in need of treatment of a disease, and more specifically, refers to a mammal such as a human or a non-human primate, a mouse, a rat, a dog, a cat, a horse, and a cow.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

The present invention provides a method of characterizing a responder to a therapeutic agent for a muscle weakness-related disease to treat a muscle weakness-related disease, the method comprising:
(S1) measuring an expression level of a fibroblast growth factor 18 (FGF18) protein or an mRNA thereof in a biological sample isolated from a subject; and
(S2) determining a responder to the therapeutic agent for a muscle weakness-related disease when the expression level of the FGF18 protein or the mRNA thereof measured in step (S1) is increased compared with that of a control group,

In the present invention, the term “increase” means at least 1%, 2%, 3%, 4%, 5%, 10% or more, for example 5%, 10%, 20%, 30%, 40%, 50%, 60%, 70%, 80%, 90% or more higher than that of the control group, and/or 0.5-fold, 1.1-fold, 1.2-fold, 1.4-fold, 1.6-fold, 1.8-fold or more higher. Specifically, it may mean an increase of 1- to 1.5-fold, 1.5- to 2-fold, 2- to 2.5-fold, 2.5- to 3-fold, 3- to 3.5-fold, 3.5- to 4-fold, 4- to 4.5-fold, 4.5- to 5-fold, 5- to 5.5-fold, 5.5- to 6-fold, 6- to 6.5-fold, 6.5- to 7-fold, 7- to 7.5-fold, 7.5- to 8-fold, 8- to 8.5-fold, 8.5- to 9-fold, 9- to 9.5-fold, 9.5- to 10-fold, or 10-fold or more compared with that of the control group, but is not limited thereto. Additionally, in the present invention, the term “increase” may have a broad meaning including a statistically significant level, but is not limited thereto. A person skilled in the art will understand that the opposite term thereof has an opposite meaning according to the above definition.

The method is characterized in that the therapeutic agent for a muscle weakness-related disease is the pharmaceutical composition of the present invention.

In the present invention, the term “subject” means a subject in need of diagnosis, treatment responsiveness, or prognosis prediction of a disease, and more specifically means a mammal such as a human or a non-human primate, mouse, rat, dog, cat, horse, or cow. Additionally, in the present invention, the term “subject” may mean a patient having high or low therapeutic responsiveness to a therapeutic agent for a muscle weakness-related disease, or a responder or non-responder to the therapeutic agent for a muscle weakness-related disease, but is not limited thereto. Additionally, the term “subject” may be used interchangeably with “individual,” but is not limited thereto.

In one embodiment of the present invention, the muscle weakness-related disease may be one or more selected from the group consisting of sarcopenia, muscular atrophy, muscle dystrophy, and cardiac atrophy, but is not limited thereto.

In the present invention, the term “responder characterization method for a therapeutic agent for a muscle weakness-related disease” means a method for determining whether a patient exhibits therapeutic responsiveness to the therapeutic agent for a muscle weakness-related disease (a pharmaceutical composition for preventing or treating a muscle weakness-related disease) of the present invention, and refers to a companion diagnostic method for determining whether to apply (administer) the therapeutic agent for a muscle weakness-related disease of the present invention to improve, prevent, and/or treat a muscle weakness-related disease in a patient with a muscle weakness-related disease. Therefore, a patient determined as a responder to the therapeutic agent for a muscle weakness-related disease according to the responder characterization method of the present invention may be expected to exhibit a therapeutic effect for a muscle weakness-related disease upon administration of the therapeutic agent for a muscle weakness-related disease.

In the present invention, the term “companion diagnostics” means one of diagnostic tests used to determine whether a particular therapeutic drug can be applied to a particular patient, and in the present invention, to determine whether a therapeutic agent for a muscle weakness-related disease (for example, a pharmaceutical composition for preventing or treating a muscle weakness-related disease according to the present invention) can be applied to a patient having a muscle weakness-related disease, the expression level of a fibroblast growth factor 18 (FGF18) protein or an mRNA thereof in a biological sample isolated from the patient may be measured as a companion diagnostic marker.

In the present invention, the term “responder to a therapeutic agent for a muscle weakness-related disease to treat a muscle weakness-related disease” may mean a patient with a muscle weakness-related disease who needs administration of the therapeutic agent for a muscle weakness-related disease of the present invention, a patient who requires continued administration of the same therapeutic agent for a muscle weakness-related disease, or a patient who is suitable for continued administration of the same therapeutic agent for a muscle weakness-related disease, but is not limited thereto.

The present invention provides a diagnostic composition for a muscle weakness-related disease, comprising, as an active ingredient, an agent for measuring an expression level of a fibroblast growth factor 18 (FGF18) protein or an mRNA of a gene encoding the FGF18 protein.

In one embodiment of the present invention, the muscle weakness-related disease may be one or more selected from the group consisting of sarcopenia, muscular atrophy, muscle dystrophy, and cardiac atrophy, but is not limited thereto.

In the present invention, the term “diagnosis” includes determining the susceptibility of a subject to a particular disease or disorder, determining whether the subject currently has the disease or disorder, determining the prognosis of a subject having the disease or disorder, or therametrics (for example, monitoring the condition of an object to provide information on therapeutic efficacy).

In one embodiment of the present invention, the muscle weakness-related disease may satisfy one or more characteristics selected from the group consisting of:
(a) decreased skeletal muscle mass;
(b) decreased one or more exercise abilities selected from the group consisting of muscle strength, muscle endurance, and maximal speed;
(c) suppressed regeneration or growth of muscle;
(d) decreased diameters of myotube cells;
(e) increased activity or proliferation of satellite cells; and
(f) decreased differentiation of satellite cells.

In the present invention, in “(a) decreased skeletal muscle mass,” the skeletal muscle may be one or more selected from the group consisting of the quadriceps (Quad), gastrocnemius (GAS), soleus (Sol), tibialis anterior (TA), and extensor digitorum longus (EDL), but is not limited thereto.

In the present invention, in “(b) decreased one or more exercise abilities selected from the group consisting of muscle strength, muscle endurance, and maximal speed,” muscle strength may be measured using a grip strength meter, muscle endurance may be measured through a Grid-Hanging test, and maximal speed may be measured through a treadmill test, but is not limited thereto.

In the present invention, in “(c) suppressed regeneration or growth of muscle,” muscle regeneration and growth may be confirmed through eMyHC, which is a marker expressed in regenerating muscle, and Laminin, which is a muscle basement membrane marker, and suppression of muscle regeneration and growth may mean a decrease in the eMyHC-positive area, but is not limited thereto.

In the present invention, in “(d) decreased diameters of myotube cells,” the decrease in myotube cell diameters may be confirmed through MyHC, but is not limited thereto.

In the present invention, in “(e) increased activity or proliferation of satellite cells,” increased activity of satellite cells may mean an increase in MyoD, which is a marker of activated satellite cells, and increased proliferation of satellite cells may mean an increase in KI67, which is a marker of proliferating cells, or an increase in PAX7, which is a marker of satellite cells, but is not limited thereto.

In the present invention, in “(f) decreased differentiation of satellite cells,” decreased differentiation of satellite cells may mean a decrease in MyoG, which is a marker of satellite cell differentiation, but is not limited thereto.

In one embodiment of the present invention, the agent for measuring a protein expression level may be an antibody specific for the FGF18 protein, but is not limited thereto.

In one embodiment of the present invention, the agent for measuring an mRNA expression level may be a probe or a primer set that specifically binds to an mRNA of FGF18, but is not limited thereto.

The present invention provides a diagnostic kit for a muscle weakness-related disease, comprising an agent for measuring a level of a fibroblast growth factor 18 (FGF18) protein or an mRNA of a gene encoding the FGF18 protein.

In one embodiment of the present invention, the kit may further comprise an instruction manual describing the method of the present invention, but is not limited thereto.

In the present invention, the term “kit” may comprise a container, an instruction manual, and an agent for measuring a level of the biomarker of the present invention. The container may serve to package the agent and may also serve to store and secure the agent. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

In one embodiment of the present invention, the kit may be one or more selected from the group consisting of a microarray, an aptamer chip kit, an ELISA (enzyme linked immunosorbent assay) kit, a blotting kit, an immunoprecipitation kit, an immunofluorescence assay kit, a protein chip kit, a reverse transcription polymerase chain reaction (RT-PCR) kit, and a quantitative real-time polymerase chain reaction (qRT-PCR) kit, but is not limited thereto.

The present invention provides a method for providing information for diagnosing a muscle weakness-related disease, comprising measuring an expression level of a fibroblast growth factor 18 (FGF18) protein or an mRNA of a gene encoding the FGF18 protein in a biological sample isolated from a subject.

In one embodiment of the present invention, the method may comprise determining that the subject has a muscle weakness-related disease when the expression level of the FGF18 protein or an mRNA of a gene encoding the FGF18 protein is increased compared with that of a normal control sample, but is not limited thereto.

In the present invention, the method for providing information for diagnosing a muscle weakness-related disease may further comprise determining that the subject has a muscle weakness-related disease when one or more levels selected from the group consisting of the following are decreased compared with those of a normal control sample, but is not limited thereto:
(S1) an expression level of a protein related to sarcomere formation and muscle contraction, or an mRNA of a gene encoding the protein, in a biological sample isolated from the subject;
(S2) an expression level of a subprotein related to actin filaments responsible for sarcomere formation and movement, or filament-based movement, or an mRNA of a gene encoding the subprotein, in a biological sample isolated from the subject; and
(S3) an expression level of a protein related to cytoskeletal signaling pathways of muscle cells, or an mRNA of a gene encoding the protein, in a biological sample isolated from the subject.

In one embodiment of the present invention, the muscle weakness-related disease may be one or more selected from the group consisting of sarcopenia, muscular atrophy, muscle dystrophy, and cardiac atrophy, but is not limited thereto.

In one embodiment of the present invention, the biological sample may be one or more selected from the group consisting of tissue, cells, whole blood, blood, serum, and plasma, but is not limited thereto.

The above-described matters of the present invention may be applied when they can be used in the screening method, the composition, the responder characterization method, the kit, and the information-providing method of the present invention, but are not limited thereto.

Hereinafter, preferred examples are provided to assist in the understanding of the present invention. However, the following examples are merely provided to facilitate the understanding of the present invention, and the scope of the present invention is not limited by the following examples.

### EXAMPLES

### Example 1.1: Confirmation of Decrease in Skeletal Muscle Mass in FGF18-Administered Group after Muscle Damage Through CTX

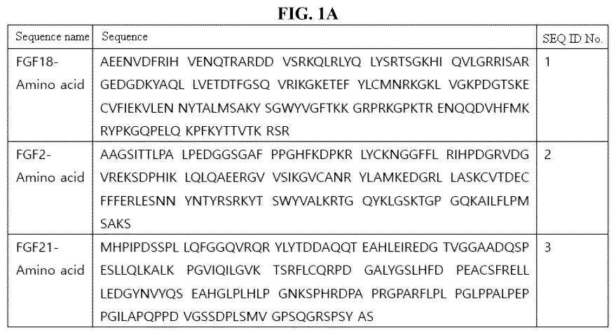

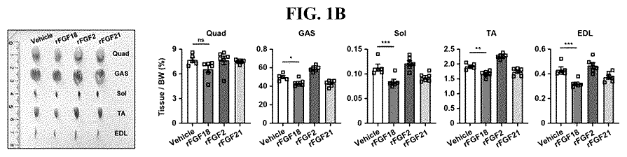

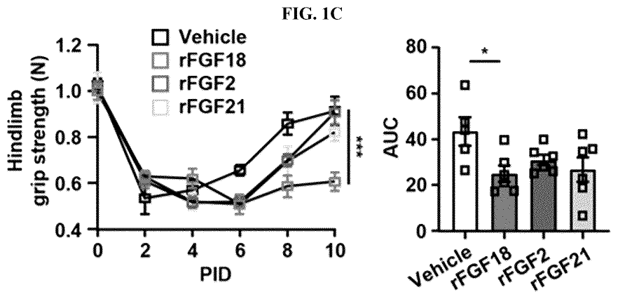

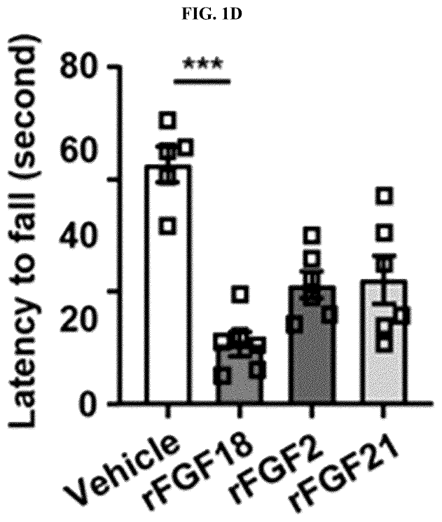

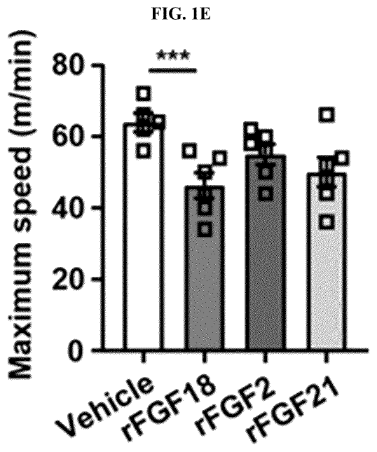

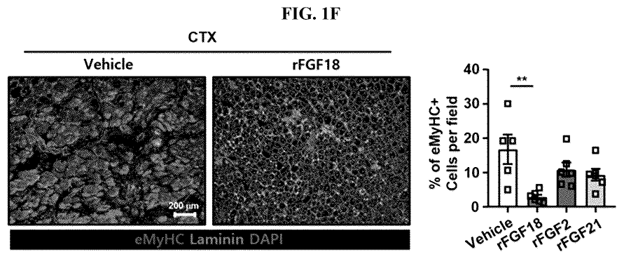

In this example, muscle damage was caused by injecting cardiotoxin (CTX) into the tibialis anterior muscle of 8-week-old male C57BL6 mice. The recombinant protein FGF18, FGF2, or FGF21 (FIG. 1A) was injected into the CTX-administered tibialis anterior muscle on days 3, 6, and 9 after muscle damage, and PBS was injected into the control. On day 10 after muscle damage, the mice were sacrificed via CO2 anesthesia, and five representative leg muscles, quadriceps (Quad), gastrocnemius (GAS), soleus (Sol), tibialis anterior (TA), and extensor digitorum longus (EDL), were collected. After the weight of each muscle was measured, the data was corrected to the mouse's body weight and graphically plotted (FIG. 1B).

As a result, it was confirmed that the skeletal muscle mass was significantly reduced in the FGF18-administered group compared to the FGF2- and FGF21-administered groups after muscle damage through CTX (FIG. 1B).

### Example 1.2: Confirmation of Decrease in Muscle Strength Recovery in FGF18-Administered Group after Muscle Damage Through CTX

In this example, hindlimb muscle strength of mice was measured and graphically plotted on days 0, 2, 4, 6, 8, and 10 after mouse muscle damage (FIG. 1C). On day 9 after damage, the Grid-Hanging test was performed to measure muscular endurance (FIG. 1D), and the maximum running speed of the mice was measured using a treadmill (FIG. 1E).

As a result, it was confirmed that the muscle strength recovery in the FGF18-administered group was significantly reduced after muscle damage through CTX (FIG. 1C), and that the muscle endurance (FIG. 1D) and maximum speed (FIG. 1E) were also significantly reduced after muscle damage through CTX.

### Example 1.3: Confirmation of Decrease in Regenerating Muscle Marker in FGF18-Administered Group after Muscle Damage Through CTX

In this example, the TA muscle tissue of a mouse was prepared into frozen sections, and cut into pieces using a cryostat. After the cut tissues were permeabilized, primary antibodies against eMyHC (Developmental Studies Hybridoma Bank, F1.652), which is a marker expressed in regenerating muscles, and Laminin (Sigma Aldrich, L9393), which is a muscle basement membrane marker, were diluted at a ratio of 1:200 in PBS containing 0.3% BSA, and dispensed onto the tissues. Thereafter, the tissues were incubated overnight at 4° C. The cross-sectional tissues of muscle were then incubated with secondary antibodies (Alexa 488 (Thermo Fisher, A-21202) and Alexa 594 (Thermo Fisher, A-21203)) for an hour at room temperature. Then, the tissues were mounted with a mounting solution containing DAPI (R&D, 918-MP-010). The stained tissue samples were visualized using a confocal microscope, and the eMyHC-positive area was analyzed and graphically plotted against the total muscle area using Image J software (FIG. 1F).

As a result, it was confirmed that the expression of eMyHC, which is a regenerating muscle marker, was lowest in the FGF18-administered group (FIG. 1F), indicating that FGF18 inhibits muscle regeneration and growth.

Therefore, it was confirmed from Examples 1.1 to 1.3 that the muscle regeneration rate and muscle ability were significantly reduced in the FGF18-administered mice.

### Example 2.1: Confirmation of Increase in KI67 in Satellite Cells Treated with FGF18

In this example, to determine the extent of satellite cell proliferation by FGF18, satellite cells isolated from 8-week-old male CT57BL6 wild-type mice were seeded on a Matrigel-coated plate. Satellite cells were treated with FGF2, FGF18, or FGF21 at a concentration of 2.5 ng/ml per well along with the control, and cultured for 4 days. At this time, the medium was replaced every 2 days. Thereafter, to perform flow cytometry analysis, the cells were detached from the plate using trypsin/streptomycin, and then centrifuged to remove the supernatant. Then, an FACS buffer was added, and a blocking process was performed at 4° C. for 10 minutes to prevent non-specific antibody binding. Afterwards, the cell surface markers Integrin alpha 7 (APC) (R&D System, FAB3518A), CD31 (APC-Cy7) (BioLegend, 102440), CD45 (BioLegend, 103113), CD11b (BioLegend, 101216), Sca1 (PE-Cy7) (BioLegend, 108113), and CD106 (PE) (BioLegend, 105713) antibodies were diluted at a ratio of 1:300 in an FACS buffer, added, and reacted at 4° C. for 30 minutes. To stain the cell proliferation marker KI67, the cells were fixed with a fixation solution, and permeabilized to allow the antibodies to penetrate into the nucleus. Then, a KI67 (Pacific Blue) (BioLegend, 652421) antibody was diluted at a ratio of 1:100 in an FACS buffer, dispensed onto the cells, and incubated on ice for 20 minutes. The proportion of the proliferated satellite cells was then measured using flow cytometry.

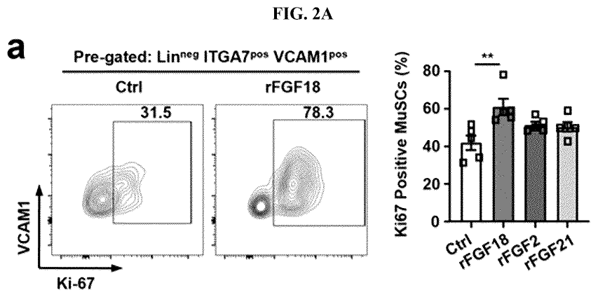

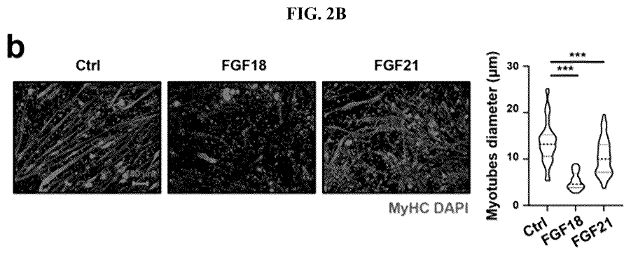

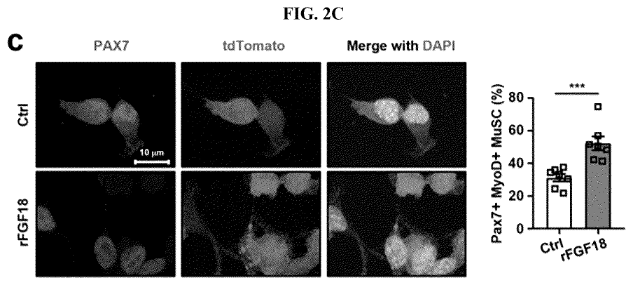

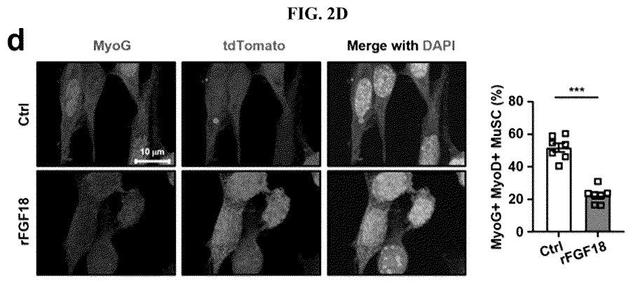

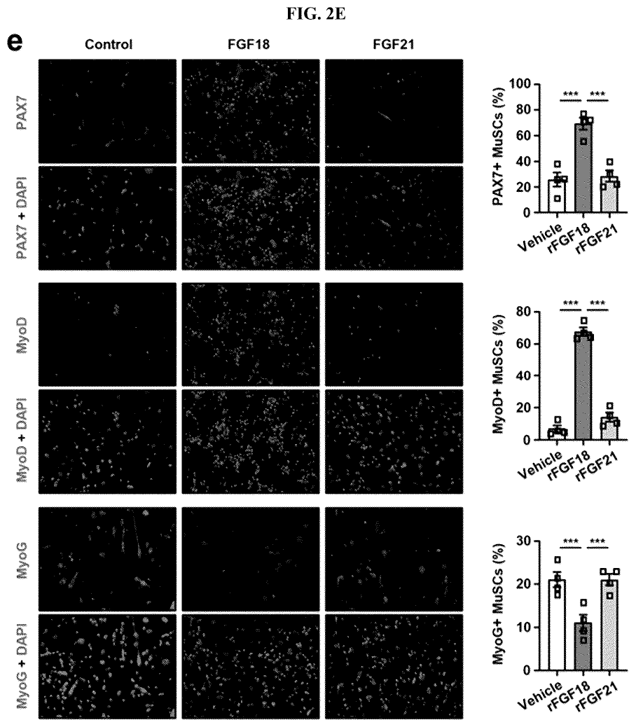

As a result, it was confirmed through the flow cytometry analysis that the satellite cell group treated with the recombinant protein FGF18 showed an increase in KI67, which is a proliferating cell marker (FIG. 2A). Therefore, this Example 2.1 suggests that the satellite cell proliferation increases when the satellite cells are treated with FGF18.

### Example 2.2: Confirmation of Decrease in Diameter in Myotube Cells Treated with FGF18

In this example, satellite cells were seeded on a Matrigel-coated plate using a DMEM medium containing 20% FBS, and then cultured at 37° C. When the cell confluency reached 70%, the medium was replaced with a DMEM medium containing 2% horse serum to induce differentiation into myotube cells. Differentiation was carried out for a total of 4 days, and the myotube cells were treated with FGF18 or FGF21 on day 2 of differentiation. On day 4 of differentiation, the cells were fixed in cold 4% paraformaldehyde for 5 minutes, and permeabilized with 0.3% Triton X-100. Thereafter, to prevent non-specific binding of antibodies, a blocking process was performed with a PBS solution containing 1% BSA, 5% serum and an MOM solution. Then, for myosin staining, a MyHC (Developmental Studies Hybridoma Bank, MF20) primary antibody was diluted at a ratio of 1:100 in PBS containing 0.3% BSA, dispensed onto the tissues, and incubated overnight at 4° C. Then, the cross-sectional tissues of muscle were incubated with a secondary antibody (Alexa 488 (Thermo Fisher, A-21202)) at room temperature for an hour. Subsequently, the tissue was mounted with a mounting solution containing DAPI. The stained tissue samples were visualized through a fluorescence microscope, and the diameters of the myotube cells was measured and graphically plotted using Image J software.

As a result, it was confirmed that the diameters of the myotube cells were significantly reduced when the differentiated myotube cells were treated with FGF18 (FIG. 2B).

### Example 2.3: Confirmation of Increased Activity and Decreased Differentiation of Satellite Cells Treated with FGF18

In this example, satellite cells were isolated from the hind limb muscles of 8-week-old male C57BL/6J-Myod1em1 (tdTomato)Utr mice, in which MyoD, which is an activated satellite cell marker, was genetically modified to exhibit tdTomato fluorescence, and seeded on a Matrigel-coated plate. The cells were treated with FGF18 at a concentration of 2.5 ng/mL along with the control, and cultured for 4 days. At this time, and the medium was replaced every 2 days. On day 4 of proliferation, the satellite cells were fixed, permeabilized, and blocked in the same manner as in Example 2.2. Thereafter, primary antibodies against PAX7 (Developmental Studies Hybridoma Bank, PAX7), which is a satellite cell marker, and MyoG (Developmental Studies Hybridoma Bank, F5D), which is a differentiation marker, were diluted at a ratio of 1:100 in PBS containing 0.3% BSA, dispensed onto the tissues, and cultured overnight at 4° C. Afterwards, the cross-sectional tissues of muscle were treated with a secondary antibody (Alexa 488 (Thermo Fisher, A-21202)), and incubated at room temperature for an hour. Then, the tissues were mounted with a mounting solution containing DAPI. The stained tissue samples were visualized using a confocal microscope, and the proportions of PAX7- and tdTomato-positive satellite cells and MyoG- and tdTomato-positive satellite cells were measured and graphically plotted using Image J software (FIGS. 2C and 2D).

Also, satellite cells were isolated from the hind limb muscles of C57BL6 wild-type mice and seeded on a Matrigel-coated plate. The cells were treated with FGF18 or FGF21 at a concentration of 2.5 ng/ml along with the control, and cultured for 4 days. At this time, the medium was replaced every 2 days. On day 4 of proliferation, the satellite cells were fixed, permeabilized, and blocked in the same manner as in Example 2.2. Thereafter, a primary antibody against the satellite cell marker PAX7 (Developmental Studies Hybridoma Bank, PAX7), the activation marker MyoD (Invitrogen, MA1-41017), or the differentiation marker MyoG (Developmental Studies Hybridoma Bank, F5D) was diluted 1:100 in PBS containing 0.3% BSA, dispensed onto the tissues, and cultured overnight at 4° C. Afterwards, the cross-sectional tissues of muscle were treated with a secondary antibody (Alexa 594 (Thermo Fisher, A-21203)) and incubated at room temperature for an hour. The tissues were then mounted with a mounting solution containing DAPI. The stained tissue samples were visualized using a confocal microscope, and the proportion of PAX7, MyoD, or MyoG-positive satellite cells was measured and graphically plotted using Image J software (FIG. 2E).

As a result, it was confirmed that in the satellite cell group treated with FGF18, PAX7, which is a satellite cell marker, and MyoD (tdTomato), which is a satellite cell activation marker, increased (FIG. 2C), but MyoG, which is a differentiation marker into myotube cells, decreased (FIG. 2D). Based on the results of staining for activation, proliferation, and differentiation markers of satellite cells, it was also confirmed that in the satellite cell group treated with FGF18, the activity and proliferation of satellite cells increased, but the differentiation of satellite cells decreased (FIG. 2E).

Therefore, it was confirmed from Examples 2.1 to 2.3 that when the satellite cells were treated with FGF18, the activity and proliferation of satellite cells increased, but the differentiation of satellite cells into myotube cells was inhibited.

### Example 3.1: Confirmation of Decreased Skeletal Muscle Exercise Ability in Mouse Model of Liver Disease

In this example, the experiment was performed after 8-week-old male C57BL6 mice were divided into three groups (Chow group, FPC group, and HFD group) by providing different diets to the mice, and the results of the analysis of the decreased skeletal muscle exercise ability were then confirmed. Specifically, the Chow group was fed a regular feed, the FPC (high fat diet with palmitic acid and cholesterol) group was fed a steatohepatitis-inducing feed (cholesterol and trans-fat) and sugar water (fructose+glucose) for 16 weeks, and the HFD (high-fat diet) group was fed a high-fat diet and sterilized tap water for 8 weeks. At the end of the experiment, the forelimb muscle strength was measured using a grip strength meter for the three groups (Chow group, FPC group, and HFD group) and the muscle endurance was measured using the Grid-Hanging test. Also, the exercise ability was measured using a treadmill test. Specifically, the treadmill test was conducted at a starting speed of 8 m/see, which was gradually increased by 2 m/min every 10 minutes, and the degree of exhaustion of the mice was recorded for a total of 120 minutes.

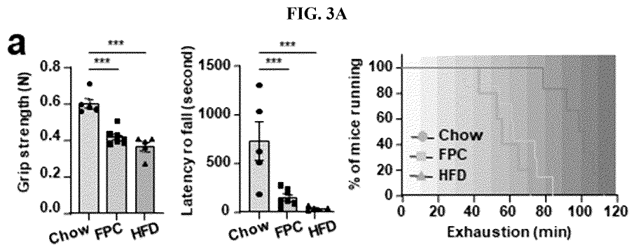

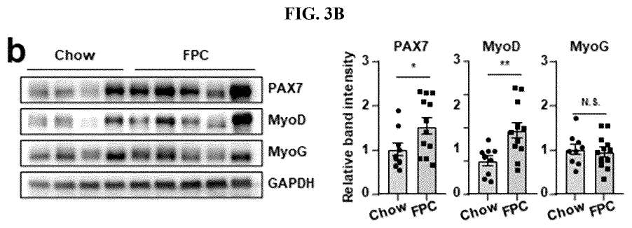

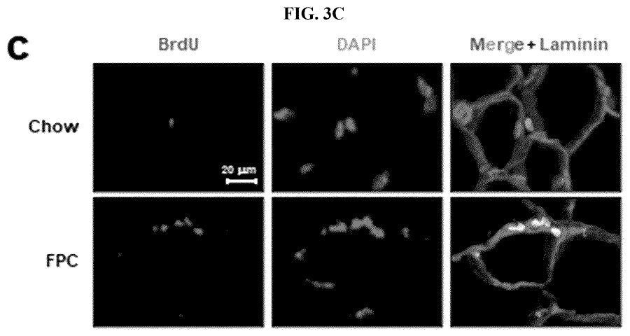

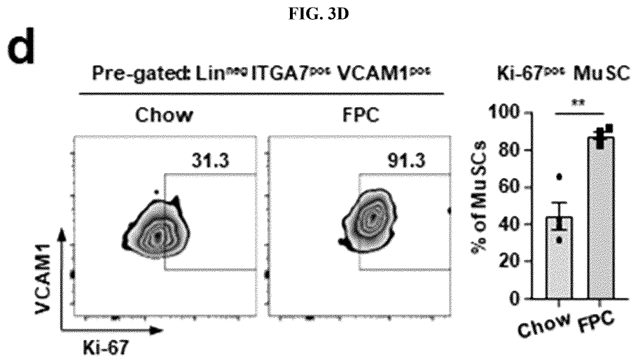

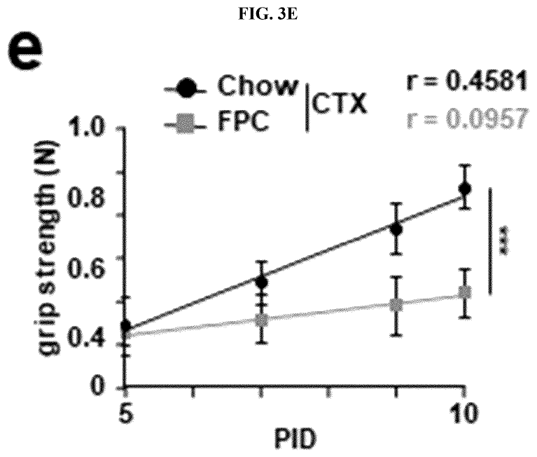

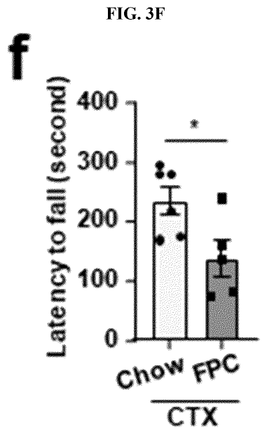

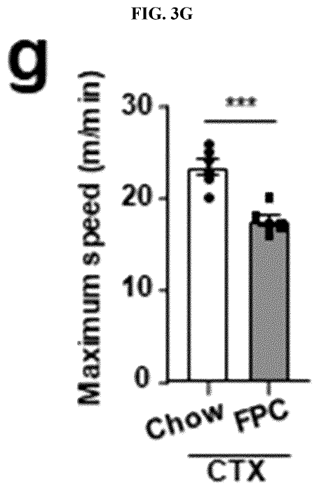

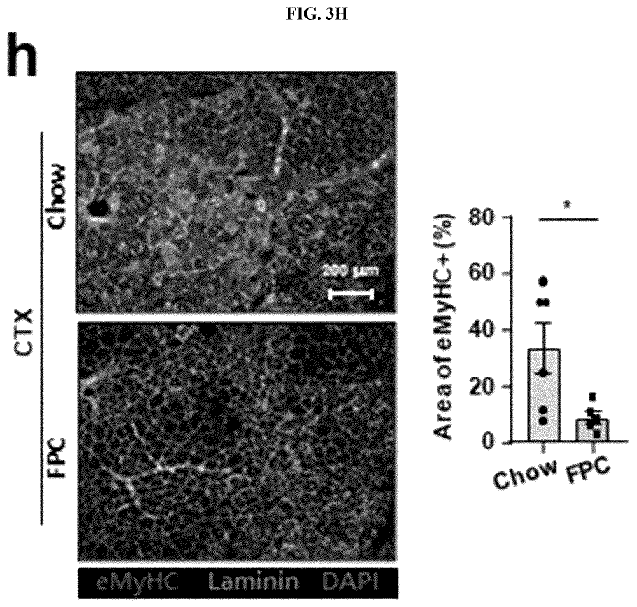

As a result, it was confirmed that the skeletal muscle exercise ability (grip, hanging, and treadmill) was reduced in the mice in the FPC group compared to the Chow group (FIG. 3A). Therefore, it was confirmed in this example that the skeletal muscle exercise ability was reduced in the mice with diet-induced metabolic dysfunction-associated steatohepatitis, which is a mouse model of liver disease.

### Example 3.2: Confirmation of Increased Expression of PAX7 and MyoD Proteins in Mouse Model of Liver Disease

In this example, the TA muscles of the mice in the Chow and FPC groups were extracted with a radio-immunoprecipitation assay (RIPA) buffer, and proteins were then separated by size using 10% gel SDS-PAGE. The separated proteins were transferred to a polyvinylidene difluoride (PVDF) membrane. The PVDF membrane was blocked at room temperature for an hour in a Tris-buffered saline-Tween 20 (TBS-T) solution supplemented with 5% skim milk, and then incubated with primary antibodies (PAX7 (Developmental Studies Hybridoma Bank, PAX7), MyoD (Invitrogen, MA1-41017), MyoG (Developmental Studies Hybridoma Bank, F5D), and GAPDH (Bioss, bs-2188R)) overnight at 4° C. Thereafter, the membrane was incubated with secondary antibodies (Bio-rad, 1706516 and 1706515) at room temperature for an hour, and luminol was dispensed onto the PVDF membrane, which was then visualized using a Fusion Solo 6X chemiluminescence system (Vilber, Collegien, France). The expression levels of the proteins were quantified using Image J software.

As a result, it was confirmed that the expression of PAX7 protein, which is a satellite cell marker, and MyoD protein, which is a satellite cell activation marker, increased in the FPC group, whereas there was no change in the expression of the MyoG protein, which is a satellite cell differentiation marker (FIG. 3B).

### Example 3.3: Confirmation of Increased Intramuscular Cell Proliferation in Mouse Model of Liver Disease

In this example, 24 hours before the end of the experiment, a bromodeoxyuridine (BrdU) pulsing solution was injected intraperitoneally into the abdominal cavities of mice. After the experiment was completed, skeletal muscles isolated from the mice were embedded in an O.C.T. compound, and prepared into frozen sections. The frozen sections were cut into 5 μm-thick muscle sections using a cryostat, and permeabilized with 0.3% Triton X-100. Thereafter, primary antibodies against BrdU (Biolegend, 370704), which is a marker for proliferating cell nuclei, and Laminin (Sigma Aldrich, L9393), which is a muscle basement membrane marker, were diluted at a ratio of 1:200 in PBS containing 0.3% BSA, dispensed onto the tissues, and cultured overnight at 4° C. Then, the cross-sectional tissues of muscle were incubated with a secondary antibody (Alexa 594 (Thermo Fisher, A-21203)) at room temperature for an hour. The tissues were mounted with a mounting solution containing DAPI. The stained tissue samples were visualized using a confocal microscope.

As a result, it was confirmed that the intramuscular cell proliferation increased in the FPC group compared to the Chow group (FIG. 3C).

### Example 3.4: Confirmation of Increased Skeletal Muscle Satellite Cell Proliferation in Mouse Model of Liver Disease

In this example, mouse skeletal muscles were collected, finely pulverized, and digested for 45 minutes at 37° C. in a shaking incubator using a digestion medium (F-10 medium containing collagenase D, dispase, and 20% FBS). The digested muscle mixture was successively filtered through 100 μm and 70 μm strainers, and the filtered solution was centrifuged. After centrifugation, the supernatant was removed, and a culture medium (F-10 medium containing 20% FBS) and satellite cell isolation beads capable of binding to cells other than satellite cells were dispensed, resuspended, and then incubated on ice for 10 minutes. The satellite cells were negatively collected using a magnet, and only the satellite cells were collected, and then centrifuged to remove the supernatant. Thereafter, a FACS buffer was added and blocked at 4° C. for 10 minutes to prevent non-specific antibody binding. Then, the cell surface markers Integrin alpha 7 (APC) (R&D System, FAB3518A), CD31 (APC-Cy7) (BioLegend, 102440), CD45 (BioLegend, 103113), CD11b (BioLegend, 101216), Sca1 (PE-Cy7) (BioLegend, 108113), and CD106 (PE) (BioLegend, 105713) antibodies were diluted at a ratio of 1:300 in a FACS buffer, added, and reacted at 4° C. for 30 minutes. To stain the cell proliferation marker KI67, the cells were fixed with a fixation solution, and permeabilized to allow the antibody to penetrate into the nucleus. Afterwards, a solution obtained by diluting a KI67 (Pacific Blue) (BioLegend, 652421) antibody at a ratio of 1:100 in a FACS buffer was dispensed onto the cells, incubated on ice for 20 minutes, and then analyzed by flow cytometry to measure the proportion of proliferated satellite cells (FIG. 3D).

As a result, it was confirmed that the proliferation of the skeletal muscle satellite cells increased in the FPC group (FIG. 3D).

### Example 3.5: Confirmation of Decreased Exercise Ability in Mouse Model of Liver Disease after Muscle Damage Through CTX

In this example, 10 days before the end of the experiment, muscle damage was caused by injecting CTX into the TA muscles of the mice in both of the Chow and FPC groups, and the hindlimb muscle strength of the mice was measured on days 5, 7, 9, and 10 after the damage (FIG. 3E). For the Chow and FPC groups, to which CTX was injected the day before the end of the experiment, their muscular endurance was measured through the Grid-Hanging test (FIG. 3F), and their maximum speed was measured using a treadmill (FIG. 3G).

As a result, it was confirmed that the exercise abilities of the FPC group were more reduced than those of the Chow group after muscle damage through CTX (FIGS. 3E to 3G).

### Example 3.6: Confirmation of Decreased Skeletal Muscle Regeneration Capacity in Mouse Model of Liver Disease after Muscle Damage Through CTX

In this example, the frozen muscle sections were permeabilized, and primary antibodies against eMyHC (Developmental Studies Hybridoma Bank, F1.652), which is a regenerating muscle marker, and Laminin (Sigma Aldrich, L9393), which is a muscle basement membrane marker, were then diluted at a ratio of 1:200 in PBS containing 0.3% BSA, dispensed onto the tissues, and incubated overnight at 4° C. Thereafter, the cross-sectional tissues of muscle were treated with secondary antibodies (Alexa 488 (Thermo Fisher, A-21202) and Alexa 594 (Thermo Fisher, A-21203)), and incubated at room temperature for an hour. Then, the tissues were mounted with a mounting solution containing DAPI (R&D, 918-MP-010). The stained tissue samples were visualized using a confocal microscope, and the eMyHC-positive area was graphically plotted against the total muscle area using Image J software (FIG. 3H).

As a result, it was confirmed that the skeletal muscle regeneration capacity was significantly reduced in the FPC group after muscle damage through CTX (FIG. 3H).

Therefore, it can be seen from Examples 3.1 to 3.6 that the activity of satellite cells increased and the skeletal muscle regeneration capacity decreased in a mouse model of liver disease, particularly a mouse model of metabolic dysfunction-associated steatohepatitis.

### Example 4: Confirmation of Increased FGF18 mRNA Expression Level in Mouse Model of Liver Disease

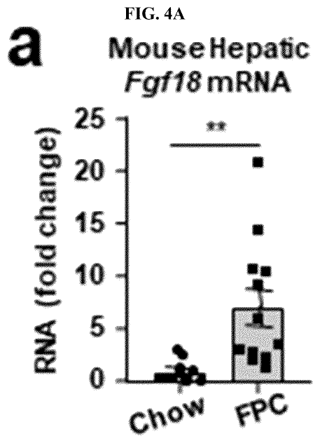

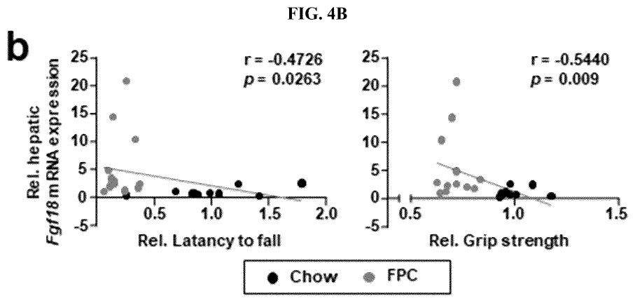

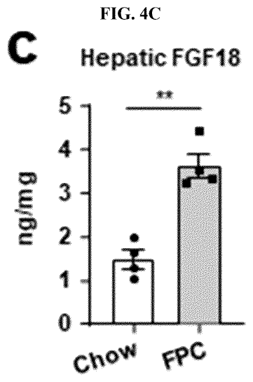

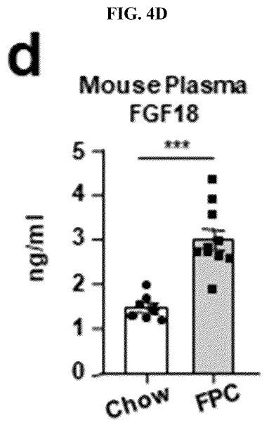

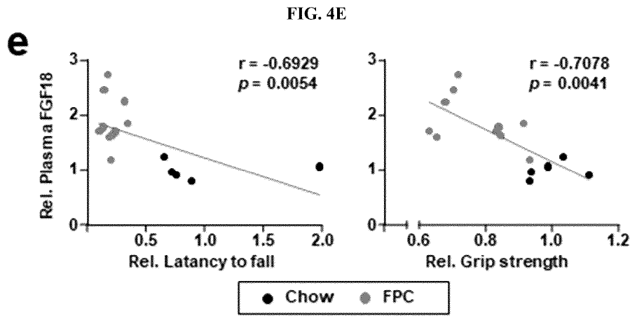

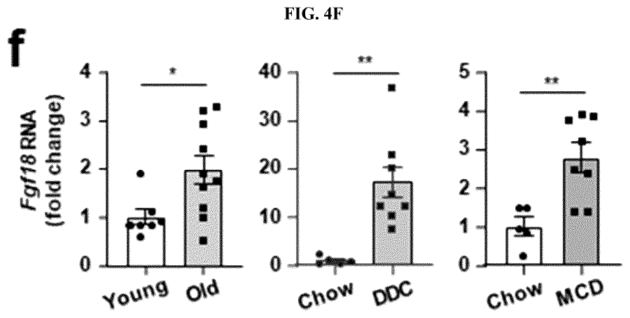

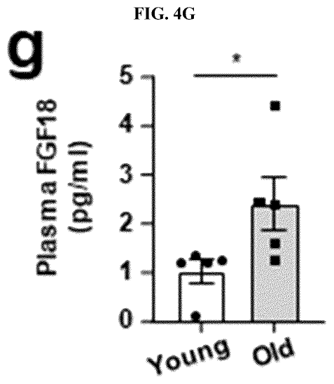

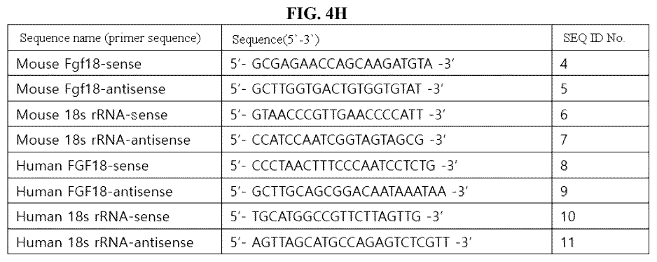

In this example, 8-week-old male C57BL6 mice were divided into two groups (Chow group and FPC group) by providing different diets to the mice, and the results were confirmed after the experiment. Specifically, the Chow group was fed a regular feed, and the FPC group was fed a steatohepatitis-inducing feed (cholesterol and trans-fat) and sugar water (fructose+glucose) for 16 weeks (FIGS. 4A to 4E).

Twenty-month-old male C57BL6 aged mice were compared with 10-week-old male C57BL6 young mice. Eight-week-old male C57BL6 mice were fed a 0.1% 3,5-diethoxycarbonyl-1,4-dihydrocollidine (DDC) feed with sterilized tap water for 4 weeks to induce a chronic liver disease, and 8-week-old male C57BL6 mice were fed a methionine/choline deficient (MCD) feed with sterilized tap water for 5 weeks to induce aging and metabolic dysfunction-associated steatohepatitis (MASH) (FIG. 4F).

After the experiment was completed, the collected mouse liver tissues were homogenized using a total RNA extraction kit and a tissue homogenizer according to the manufacturer's protocol. The purified RNA was converted to cDNA using a cDNA reverse transcription kit according to the manufacturer's protocol, and real-time PCR was performed to determine the gene expression level of fibroblast growth factor 18 (FGF18). The mRNA level was expressed as a fold change, which was derived and graphically plotted using the 2−ΔCt equation, which is the Ct value of the target gene minus the Ct value of a housekeeping gene such as 18S rRNA (FIGS. 4A and 4F). The correlation of the measured mouse grip strength scores and grid-hanging test scores with the mRNA level of Fgf18 or the concentration of the blood FGF18 protein was analyzed to confirm the slope and significance (FIGS. 4B and 4E). An ELISA experiment was performed to determine the protein level of FGF18 in the liver or blood. Specifically, a frozen liver tissue was homogenized with a mixture of protease inhibitor and RIPA buffer using a homogenizer, and then centrifuged to collect the supernatant. Mouse blood was collected in an EDTA tube, and centrifuged at 4° C. for 10 minutes to separate plasma. A concentration of the FGF18 protein was measured using an ELISA kit according to the manufacturer's protocol, and the absorbance was measured at 450 nm to confirm the protein level of FGF18 in mouse liver and blood (FIGS. 4C, 4D, and 4G). The primer sequences used for real-time PCR are shown in FIG. 4H.

As a result, it was confirmed that the mRNA level of FGF18 in the liver of the FPC group significantly increased (FIG. 4A), that the mRNA expression of FGF18 in the liver showed an inverse correlation with the skeletal muscle exercise ability (FIG. 4B), and that the protein level of FGF18 in the liver of the FPC group significantly increased compared to the Chow group (FIG. 4C).

It was confirmed that the protein level of circulating FGF18 in the blood of the FPC group increased (FIG. 4D), and that the protein level of circulating FGF18 in the blood showed an inverse correlation with the skeletal muscle exercise ability (FIG. 4E).

Also, it was confirmed that the mRNA level of FGF18 in the liver increased in aging, chronic liver disease, and aging and metabolic dysfunction-associated steatohepatitis (FIG. 4F), and that the protein level of circulating FGF18 in the blood of aging mice increased (FIG. 4G).

### Example 5.1: Confirmation of Increased Expression of FGFR4 in Satellite Cells of FPC Group

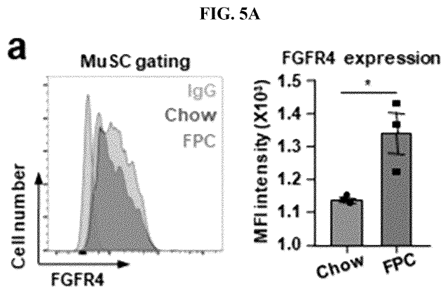

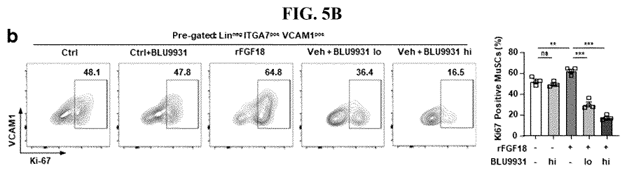

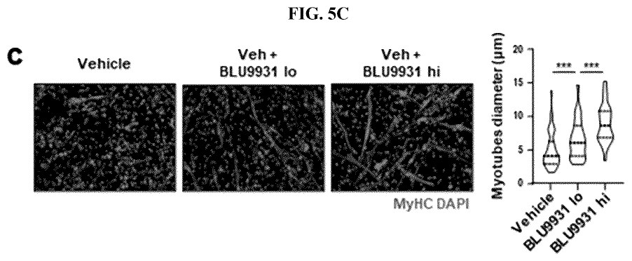

In this example, mouse skeletal muscles were collected, finely pulverized, and digested in a digestion medium (F-10 medium containing collagenase D, dispase, and 20% FBS) at 37° C. for 45 minutes in a shaking incubator. The digested muscle mixture was filtered sequentially through 100 μm and 70 μm strainers, and the filtered solution was centrifuged. The supernatant was removed, a culture medium (F-10 medium containing 20% FBS) was added, and satellite cell isolation beads, which may bind to cells other than satellite cells, were dispensed, resuspended, and then incubated on ice for 10 minutes. Satellite cells were negatively selected using a magnet to collect only the satellite cells, and then centrifuged to remove the supernatant. Thereafter, a FACS buffer was added, and blocking was performed at 4° C. for 10 minutes to prevent non-specific antibody binding. Then, the cell surface markers CD334 (PerCP-Cy5.5) (Miltenyi biotec, 130-124-928), Integrin alpha 7 (APC) (R&D system, FAB3518A), CD31 (APC-Cy7) (BioLegend, 102440), CD45 (BioLegend, 103113), CD11b (BioLegend, 101216), Sca1 (PE-Cy7) (BioLegend, 108113), and CD106 (PE) (BioLegend, 105713) antibodies were diluted at a ratio of 1:300 in a FACS buffer, reacted at 4° C. for 30 minutes, and then analyzed by flow cytometry to determine the expression level of fibroblast growth factor receptor 4 (FGFR4) in the satellite cells (FIG. 5A).

As a result, it was confirmed that the expression of FGFR4, which is an FGF18 receptor, in the satellite cells significantly increased in the FPC group (FIG. 5A).

### Example 5.2: Identification of FGFR4 Inhibitor that Reduces Satellite Cell Proliferation Increased by Treatment with FGF18

In this example, to confirm the proliferation of satellite cells by FGF18 and the inhibitory effect of FGF18 according to treatment with an FGFR4 inhibitor, satellite cells isolated from 8-week-old male CT57BL6 wild-type mice were seeded on a Matrigel-coated plate. The satellite cells were cultured by treating each well with FGF18 at a concentration of 2.5 ng/mL along with the control. On day 2 of culture, the FGFR4 inhibitor BLU9931 (C₂₆H₂₂C₁₂N₄O₃) (MedChemExpress) was treated at a low dose (5 μM) or high dose (20 μM) for 2 days. Satellite cell culturing was performed for a total of 4 days. Thereafter, the cells were harvested and blocked to perform flow cytometry analysis. Next, the cell surface markers Integrin alpha 7 (APC) (R&D System, FAB3518A), CD31 (APC-Cy7) (BioLegend, 102440), CD45 (BioLegend, 103113), CD11b (BioLegend, 101216), Sca1 (PE-Cy7) (BioLegend, 108113), and CD106 (PE) (BioLegend, 105713) antibodies were diluted at a ratio of 1:300 in a FACS buffer, and reacted at 4° C. for 30 minutes. Then, the cells were fixed with a fixation solution to stain the cell proliferation marker KI67, and then permeabilized to allow the antibodies to penetrate into the nucleus. Afterwards, a solution prepared by diluting a KI67 (Pacific Blue) antibody in a FACS buffer at a ratio of 1:100 was dispensed onto the cells, and then incubated on ice for 20 minutes. Then, the proportion of the proliferated satellite cells was measured using flow cytometry (FIG. 5B).

As a result, it was confirmed that the increased satellite cell proliferation by the treatment with FGF18 was reduced when co-treated with BLU9931, which is an FGFR4 inhibitor, and that this reduction was dose-dependent (FIG. 5B).

### Example 5.3: Identification of FGFR4 Inhibitor that Increases Diameters of Myotube Cells Treated with FGF18

In this example, satellite cells isolated from 8-week-old male C57BL6 wild-type mice were seeded on a Matrigel-coated plate using a DMEM medium containing 20% FBS, and cultured at 37° C. When the cell confluency reached 70%, the medium was replaced with a DMEM medium containing 2% horse serum to induce differentiation into myotube cells. The differentiation process was performed for a total of 4 days. On day 2 of differentiation, the myotube cells were treated with 2.5 ng/ml of FGF18, 2.5 ng/ml of FGF18 and 5 μM of BLU9931, or 2.5 ng/ml of FGF18 and 20 μM of BLU9931, depending on the experimental groups. On day 4 of differentiation, the cells were fixed with cold 4% paraformaldehyde for 5 minutes, and permeabilized with 0.3% Triton X-100. Thereafter, to prevent non-specific antibody binding, a blocking process was performed with a PBS solution containing 1% BSA, 5% serum and an MOM solution. Then, to stain myosin, a MyHC (Developmental Studies Hybridoma Bank, MF20) primary antibody was diluted at a ratio of 1:100 in PBS containing 0.3% BSA, dispensed onto the tissues, and incubated overnight at 4° C. Afterwards, the cross-sectional tissues of muscle were incubated with a secondary antibody (Alexa 488 (Thermo Fisher, A-21202)) at room temperature for an hour. Then, the tissues were mounted with a mounting solution containing DAPI. The stained tissue samples were visualized through a fluorescence microscope, and the diameters of the myotube cells were measured and diagrammed using Image J software (FIG. 5C).

As a result, it was confirmed that when the FGF18-treated myotube cells (vehicle) were treated with a low dose (Veh+BLU9931 lo) or high dose (Veh+BLU9931 hi) of an FGFR4 inhibitor, the diameters of myotube cells increased in a dose-dependent manner (FIG. 5C).

Therefore, it was confirmed from Examples 5.1 to 5.3 that the diameters of myotube cells treated with FGF18 increased when FGFR4 was inhibited.

### Example 6: Confirmation of Decreased Expression of Genes Associated with Formation of Skeletal Muscle Cell Structure in Satellite Cells Treated with FGF18

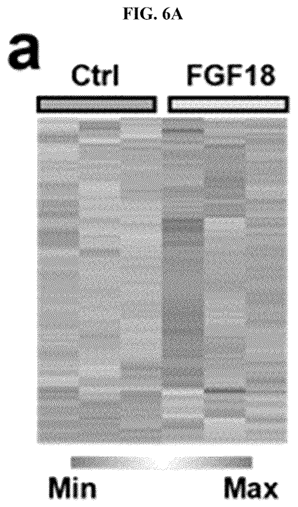

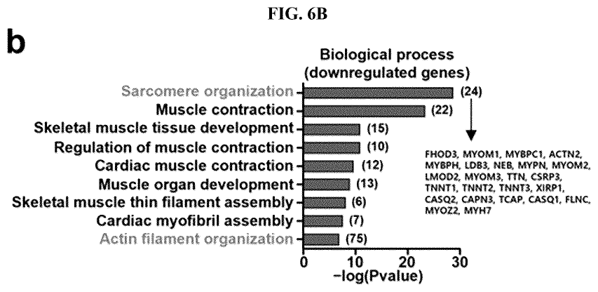

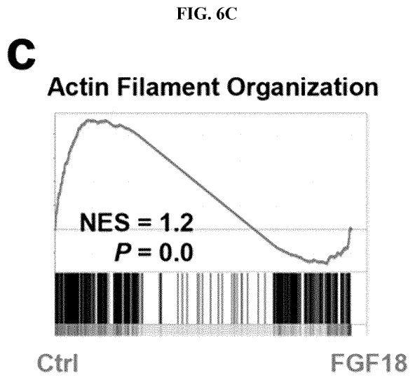

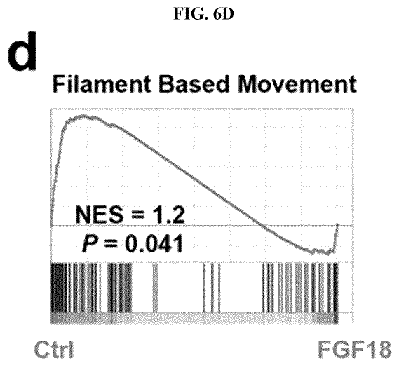

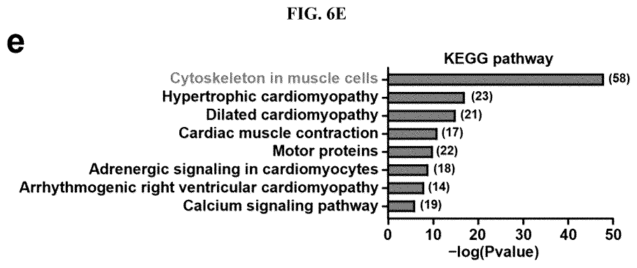

In this example, to analyze genetic changes in satellite cells induced by FGF18, satellite cells isolated from 8-week-old male CT57BL6 wild-type mice were seeded on a Matrigel-coated plate. The satellite cells were cultured for 4 days together with the control (Ctrl) and the group treated with FGF18 at a concentration of 2.5 ng/mL. At this time, the medium was replaced every 2 days. On day 4 of culture, total RNA was isolated using Trizol, and RNA quantification was performed. To construct a gene library and perform sequencing, the CORALL RNA-Seq V2 Library Prep kit was used, and mRNA isolation was performed using the poly(A) siRNA Selection kit. The isolated mRNA was used for cDNA synthesis and shearing according to the manufacturer's protocol, and then concentrated through PCR. Thereafter, the library was sequenced using a TapeStation HS D1000 Screen tape to evaluate the average fragment size, and sequenced using a Real-Time PCR system to quantify the library. FastQC was used for quality control of the sequencing data. The sequenced reads were trimmed for adapter sequences and filtered using BBDuk. The filtered outputs were mapped to the reference genome using STAR. Read quantification was processed using HTSeq-count. Read counts were processed using a TMM+CPM normalization method using the Python “conorm” package, and gene ontology was identified using the DAVID functional annotation tool (NIH) and gene set enrichment analysis (GSEA). A heatmap for gene expression was generated using the phantasus package (https://artyomovlab.wustl.edu/phantasus/) (FIGS. 6A to 6E).

As a result, it was confirmed that the gene expression changed in the satellite cell group treated with FGF18, and also confirmed that the skeletal muscle mass and function were improved through inhibition of FGF18 signaling. Specifically, the global transcriptomics of satellite cells treated with FGF18 were investigated (FIGS. 6A to 6D). Based on the results of investigation, it can be seen that the treatment of satellite cells with FGF18 downregulated various mRNA levels, including genes associated with sarcomere formation and muscle contraction (FIG. 6B). Also, it can be seen that downstream genes associated with actin filaments (FIG. 6C) and filament-based movements (FIG. 6D), which are responsible for sarcomere formation and movement, also decreased with the treatment with FGF18. Consistent with this, KEGG analysis results showed that the treatment with FGF18 suppressed mRNA transcription of genes associated with the cytoskeleton in muscle cells signaling pathway (FIG. 6E). Therefore, this suggests that the genes involved in the formation of skeletal muscle cell structures are downregulated when the cells are treated with FGF18.

### Example 7: Confirmation of Improved Muscle Mass Through Blockade of FGF18 Signaling in In Vivo Mouse Model

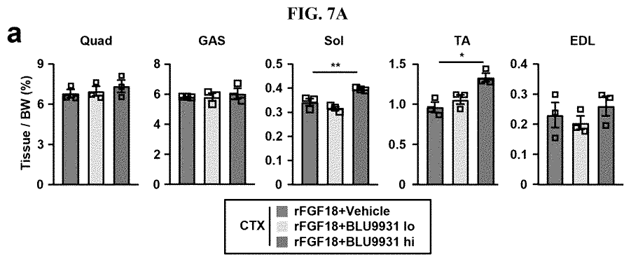

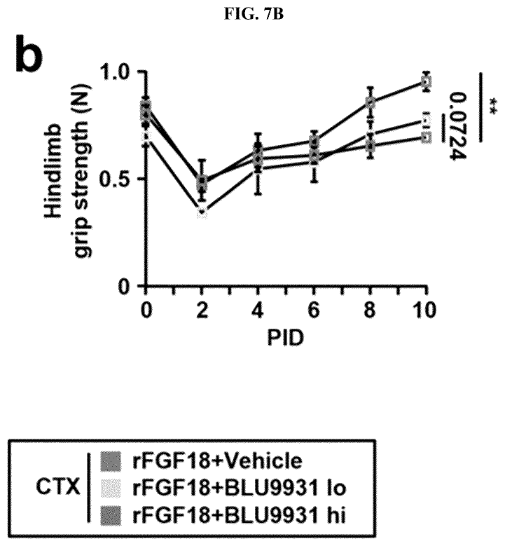

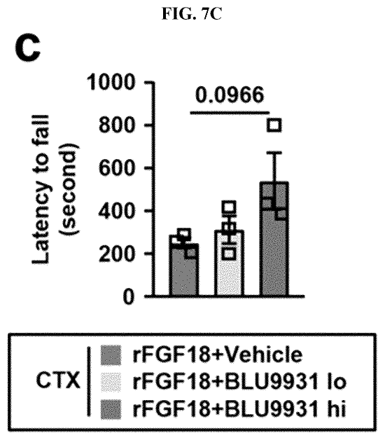

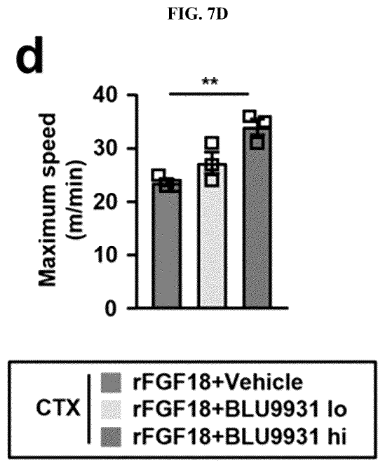

CTX was injected into the TA muscles of 8-week-old male C57BL6 mice to induce muscle damage. On days 2, 4, 6, and 8 after muscle damage, recombinant protein FGF18 was injected into the TA muscles to which CTX had been administered. BLU9931, which is an FGFR4 inhibitor, was divided into low-dose (0.1 mg/kg, rFGF18+BLU9931 lo) and high-dose (1 mg/kg, rFGF18+BLU9931 hi) groups, and both were administered together with FGF18. After the experiment was completed, the mice were sacrificed using CO2 anesthesia, and the muscle tissues of the quadriceps (Quad), gastrocnemius (GAS), soleus (Sol), TA, and EDL were collected. The weight of each muscle was measured, corrected to body weight, and graphically plotted (FIG. 7A). On days 0, 2, 4, 6, 8, and 10 after mouse muscle damage, the hind limb muscle strength of the mice was measured, and the change patterns were graphically plotted (FIG. 7B). On day 9 after damage, the grid-hanging test was performed to measure muscle endurance (FIG. 7C), and the maximum running speed of the mice was measured using a treadmill (FIG. 7D).

As a result, it was confirmed that the FGF18 signaling blockage enhanced muscle regeneration in an in vivo mouse model. Specifically, to confirm the muscle recovery effect through inhibition of FGF18, an FGFR4 inhibitor (BLU9931) was administered to the TA muscles of mice after CTX damage. As a result, it was confirmed that the muscle mass in the TA and Sol muscles significantly improved (FIG. 7A).

Also, muscle function was measured to determine whether the muscle regeneration reduced by FGF18 administration after muscle damage was restored by inhibition of the FGF18 signaling pathway. As a result, it was confirmed that the hindlimb muscle strength in the mice gradually improved compared to the FGF18+Vehicle group (FIG. 7B). Also, as seen from the Grid-Hanging test results, a trend toward improved muscle endurance was observed (FIG. 7C) and the maximum running speed was also improved (FIG. 7D).

Therefore, this suggests that the inhibition of FGF18 signaling through the FGFR4 inhibitor may have an improvement effect on muscle regeneration in a dose-dependent manner.

The foregoing description of the present invention is intended for illustrative purposes, and it will be understood by those skilled in the art that various modifications can be made thereto in other specific forms without departing from the technical spirit or essential features of the present invention. Therefore, the embodiments described above should be understood as illustrative in all respects and not restrictive.

1.-20. (canceled)

21. A method for treating a muscle weakness-related disease comprising administering to a subject in need thereof, a composition comprising a pharmaceutically effective amount of an agent that inhibits the expression of a fibroblast growth factor receptor 4 (FGFR4) gene or the activity of FGFR4 protein.

22. The method of claim 21, wherein the agent binds to the FGFR4 to inhibit a fibroblast growth factor 18 (FGF18) signaling pathway mediated by the FGFR4.

23. The method of claim 21, wherein the muscle weakness-related disease is one or more selected from the group consisting of sarcopenia, muscle atrophy, muscle dystrophy, and cardiac atrophy.

24. The method of claim 21, wherein the composition satisfies one or more characteristics selected from the group consisting of:
(a) reducing the proliferation of satellite cells;
(b) increasing the diameters of myotube cells;
(c) increasing skeletal muscle mass; and
(d) increasing one or more exercise abilities selected from the group consisting of muscle strength, muscle endurance, and maximal speed.

25. The method of claim 21, wherein the agent that inhibits the expression of the gene is one or more selected from the group consisting of miRNA, siRNA, shRNA, a ribozyme, a DNAzyme, peptide nucleic acid (PNA), and an antisense oligonucleotide which complementarily bind to the mRNA of the FGFR4 gene, and the agent that inhibits the activity of the FGFR4 protein is one or more selected from the group consisting of a peptide, an antibody, an aptamer, and a compound which specifically bind to the FGFR4 protein.

26. A method of characterizing a responder to a therapeutic agent for a muscle weakness-related disease and treating a muscle weakness-related disease, comprising:
(S1) measuring an expression level of a FGF18 protein or an mRNA thereof in a biological sample isolated from a subject;
(S2) determining a responder to the therapeutic agent for a muscle weakness-related disease when the expression level of the FGF18 protein or mRNA thereof measured in step (S1) increases, compared with a control; and
(S3) administering to the responder a pharmaceutically effective amount of the composition used in the method of claim 25 to treat a muscle weakness-related disease.

27. The method of claim 26, wherein the muscle weakness-related disease is one or more selected from the group consisting of sarcopenia, muscle atrophy, muscle dystrophy, and cardiac atrophy.

28. The method of claim 26, wherein the muscle weakness-related disease satisfies one or more characteristics selected from the group consisting of:
(a) decreased skeletal muscle mass;
(b) decreased exercise ability selected from the group consisting of muscle strength, muscle endurance, and maximal speed;
(c) inhibited muscle regeneration or growth;
(d) decreased diameters of myotube cells;
(e) increased activity or proliferation of satellite cells; and
(f) decreased differentiation of satellite cells.

29. The method of claim 26, wherein the biological sample is one or more selected from the group consisting of a tissue sample, a cells sample, a whole blood sample, a blood sample, a serum sample, and a plasma sample.

30. A method for treating a muscle weakness-related disease, comprising:
(S1) measuring an expression level of a FGF18 protein or an mRNA of a gene encoding the protein in a biological sample isolated from a subject;
(S2) determining that a subject having an increased expression level of the FGF18 protein or an mRNA of a gene encoding the protein, as compared to a normal control sample, is suffering from a muscle weakness-related disease; and
(S3) treating the subject suffering from the muscle weakness-related disease.

31. The method of claim 30, wherein the muscle weakness-related disease is one or more selected from the group consisting of sarcopenia, muscle atrophy, muscle dystrophy, and cardiac atrophy.

32. The method of claim 30, wherein the muscle weakness-related disease satisfies one or more characteristics selected from the group consisting of:
(a) decreased skeletal muscle mass;
(b) decreased exercise ability selected from the group consisting of muscle strength, muscle endurance, and maximal speed;
(c) inhibited muscle regeneration or growth;
(d) decreased diameters of myotube cells;
(e) increased activity or proliferation of satellite cells; and
(f) decreased differentiation of satellite cells.

33. The method of claim 30, wherein the biological sample is one or more selected from the group consisting of a tissue sample, a cells sample, a whole blood sample, a blood sample, a serum sample, and a plasma sample.

34. The method of claim 30, wherein step (S3) of treating the subject comprises administering to the subject a composition comprising a pharmaceutically effective amount of an agent that inhibits the expression of a fibroblast growth factor receptor 4 (FGFR4) gene or the activity of FGFR4 protein.
